SEQUENCE LISTING (1 sequence)

SEQ ID NO: 1 - protein, 609 aa, Homo sapiens [sequence omitted]

(19) United States
(12) Patent Application Publication
(10) Pub. No.: US 2026/0338798 A1
(43) Pub. Date: Oct. 29, 2026

(54) USE OF ALBUMIN ISOFORMS PROFILES FOR THE CHARACTERIZATION OF THE ETIOLOGY AND SEVERITY OF LIVER INJURIES

(71) Applicants: INSTITUT NATIONAL DE LA SANTÉ ET DE LA RECHERCHE MÉDICALE, Paris (FR); CENTRE HOSPITALIER UNIVERSITAIRE DE LIMOGES, Limoges (FR); UNIVERSITÉ DE LIMOGES, Limoges (FR)

(72) Inventors: Souleiman EL BALKHI, Limoges (FR); François-Ludovic SAUVAGE, Limoges (FR); Pierre MARQUET, Limoges (FR); Franck SAINT-MARCOUX, Limoges (FR); Mohamed-Ali RAHALI, Les Abymes (FR)

(21) Appl. No.: 19/111,402

(22) PCT Filed: Oct. 6, 2023
(86) PCT No.: PCT/EP2023/077721

§ 371 Date: Mar. 13, 2025

(30) Foreign Application Priority Data
Oct. 7, 2022 (EP) 22306513.7

(51) Int. Cl. G01N 33/68; A61K 31/19; A61K 31/522; A61K 31/56; A61K 45/06; G16C 20/20

(52) U.S. Cl. CPC G01N 33/6893; A61K 31/19; A61K 31/522; A61K 31/56; A61K 45/06; G16C 20/20; G01N 2333/76; G01N 2800/085; G01N 2800/56; G01N 2800/60; G01N 2800/7052

(57) ABSTRACT

The inventors hypothesized that each type of liver injury can be revealed by a specific profile of HSA posttranslational modifications. Therefore, the aim of inventors was to study the pattern of albumin isoforms in rats intoxicated with acetaminophen (APAP), ethanol, and CCI₄. The second objective was to explore the potential of these isoforms as biomarkers of liver specific injuries. The results demonstrate that albumin posttranslational modifications (Alb-PTM) occur very early during the course of liver injuries induced by hepatotoxic substances. In 3 animal models, native albumin started to decrease in favor of other isoforms 24 hours after the administration of APAP, ethanol or CCI₄. Interestingly, the nature and the intensity of isoforms were different depending on the hepatotoxic substance. In a cohort of cirrhotic patients, the inventors were able to identify up to 14 albumin isoforms, all of which were also present in control patients. However, the inventors observed that the increase in the HSA-DA isoform was specific to patients with cirrhosis due to alcohol abuse, HSA+SGGS and HSA+2Glyc were increased specifically in NASH patients, and HSA-DA+Cys with HSA+SO2H were increased only in patients with the mixed form. In addition, we did not observe a specific isoform able to clearly discriminate the different stages of liver disease, but principal component analysis of the MS dataset perfectly separated cirrhosis patients with different Child-Pugh scores and control patients. The present invention thus relates to the use of albumin isoforms profiles for the characterization of the etiology and severity of liver injuries.

### FIELD OF THE INVENTION

The present invention is in the field of medicine, in particular hepatology.

### BACKGROUND OF THE INVENTION

The pathogenesis of liver injuries is complex and involves numerous cellular, molecular, immune and hormonal disturbances (1). This makes the diagnosis of liver diseases a challenging process. The intricacy of these disturbances has led to the examination of a multitude of potential serum biomarkers that reflect underlying disease through cellular pathways, including hepatocellular apoptosis, inflammation or oxidative stress. However, despite the utility of these serum biomarkers (called ‘conventional liver biomarkers’ thereafter) in the assessment of liver injuries and chronic liver diseases, they have limited usefulness in the early stages of liver injuries (2-4).

The use of process-related biomarkers has evolved to the use of panels of biomarkers in order to reflect a bigger picture of events occurring in the hepatocytes and/or in the organ tissues. Currently, because of their lack of sensitivity and specificity when taken individually, serum markers are now gathered in panel tests, sometimes together with interpretation algorithms (Fib4, FibroMeter, FibroTest, FIBROSpect, Hepascore . . . ) or with imaging tests (FibroMeter-VCTE algorithm) to help diagnosing and staging fibrosis and/or cirrhosis. However, it has been recently argued that these tests “show limited accuracy in individual patients and do not reflect disease progression or treatment response and do not provide a mechanistic understanding of injury patterns”(3). Other biomarkers, such as molecules involved in the fibrosis process including protein-based biomarkers, microRNA or collagens, have been extensively studied. Although some are promising, they have not yet demonstrated the diagnostic or predictive performances required for early dysfunctions or for mid- or long-term liver injuries (5). Therefore, noninvasive (or less invasive), simple, sensitive and specific biomarkers for the early detection of dysfunctions able to lead to liver failure are still needed.

Recently, human serum albumin (HSA) isoforms have gained great interest as biomarkers of advanced liver diseases (6-16). Due to the exclusive synthesis of HSA by liver cells, its great chemical reactivity and its abundance (60% of all plasma proteins), it is now clear that not only the quantity, but also the quality of HSA may reflect liver dys/functions. HSA may undergo several posttranslational modifications including truncations, acetylation, cysteinylation, homocysteinylation, glutathionylation, glycation, nitrosylation, nitration, phosphorylation and oxidation (17). The clinical relevance of some of these modifications has been recently investigated in advanced liver diseases (15, 17). The oxidation of the Cys34 residue has been the most studied. It was characterized on the basis of the redox state of Cys34 as follows: (i) Human mercaptalbumin (HMA), the reduced HSA (70-80% of total HSA in healthy subjects), (ii) Nonmercaptalbumin 1 (HNA1), a reversibly oxidized form (20-30%) and (iii) Nonmercaptalbumin 2 (HNA2) the irreversible oxidized form of albumin (<5%) (17). The increase in HNA1 and HNA2 has been well documented in end-stage liver pathologies, with a progressive increase of these isoforms in severe cirrhosis correlated to a very high short-term mortality (9, 14, 15). Interestingly, it has been reported that HNA1 plays a pejorative role in decompensated cirrhosis (18) while native HSA has a protective role by reducing the proinflammatory environment present in patients with acutely decompensated cirrhosis (19). At present, it is not fully established whether other albumin isoforms are mostly inactive or possess some not yet characterized biological properties. Structural alterations involving sites others than Cys34 were reported. Indeed, N- or C-terminal truncated as well as glycated forms were found in plasma samples from patients with acutely decompensated cirrhosis or severe alcoholic hepatitis (9). Most importantly, these findings suggest that HSA modifications are directly related to liver injuries and that HSA isoforms are very likely produced because of the chemical environment into the hepatocytes.

This bundle of arguments strongly suggest that albumin modifications detected in blood may reflect the dys/function of hepatocytes and represent a versatile tool for the diagnosis and the prognosis of liver injuries and/or diseases.

### SUMMARY OF THE INVENTION

The present invention is defined by the claims. In particular, the present invention relates to the use of albumin isoforms profiles for the characterization of the etiology and severity of liver injuries.

### DETAILED DESCRIPTION OF THE INVENTION

The inventors hypothesized that each type of liver injury can be revealed by a specific profile of HSA posttranslational modifications. Since the cellular chemical environment at the origin of liver injuries is different, this logically should lead to the apparition of specific HSA isoform patterns in blood. Since HSA is continuously synthesized in hepatocytes and secreted into the bloodstream, the second hypothesis is that HSA modifications occur at early stages of cell injuries. Therefore, the aim of inventors was to study the pattern of albumin isoforms in rats intoxicated with acetaminophen (APAP), ethanol, and CCl₄. The second objective was to explore the potential of these isoforms as biomarkers of liver specific injuries. The results demonstrate that albumin posttranslational modifications (Alb-PTM) occur very early during the course of liver injuries induced by hepatotoxic substances. In 3 animal models, native albumin started to decrease in favor of other isoforms 24 hours after the administration of APAP, ethanol or CCl₄. Interestingly, the nature and the intensity of isoforms were different depending on the hepatotoxic substance. In a cohort of cirrhotic patients, the inventors were able to identify up to 14 albumin isoforms, all of which were also present in control patients. However, the inventors observed that the increase in the HSA-DA isoform was specific to patients with cirrhosis due to alcohol abuse, HSA+SGGS and HSA+2Glyc were increased specifically in NASH patients, and HSA-DA+Cys with HSA+SO2H were increased only in patients with the mixed form. In addition, we did not observe a specific isoform able to clearly discriminate the different stages of liver disease (data not shown), but principal component analysis of the MS dataset perfectly separated cirrhosis patients with different Child-Pugh scores and control patients.

### MAIN DEFINITIONS

As used herein, the term “subject” as used herein refers to any mammal organism. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

As used herein, the term “injury” refers to any damage that directly or indirectly affects the normal functioning. An insult may have a variety of causes including, but not limited to physiological injuries, chemical injuries or physical injuries. The term encompasses acute and chronic injuries. As used herein, the term “acute injury” includes injuries that have recently occurred. For example, an acute injury may have very recently occurred, may have occurred within an hour or less, may have occurred within a day or less, may have occurred within a week or less, or may have occurred within two weeks or less. As used herein, the term “chronic injury” is an injury that has persisted for a period of time. For example, a chronic injury may have occurred more than two weeks ago, may have occurred more than three weeks ago, may have occurred more than two months ago, or may have occurred more than three months ago.

As used herein, the term “liver injury” refers to a state in which the liver function is decreased relative to a normal state. Hepatic dysfunction is characteristic of liver diseases. A number of acute or chronic pathological conditions leads to liver injury. These include, but are not limited to liver abscess, liver cancer, either primary or metastatic, cirrhosis, such as cirrhosis caused by the alcohol consumption or primary biliary cirrhosis, amebic liver abscess, autoimmune hepatitis, biliary atresia, coccidioidomycosis disseminated, portal hypertension hepatic infections (such as hepatitis A virus, hepatitis B virus, hepatitis C virus, hepatitis D virus, or hepatitis E virus), hemochromatosis, hepatocellular carcinoma, pyogenic liver abscess, Reye's syndrome, sclerosing cholangitis, Wilson's disease, drug induced hepatotoxicity, or fulminant or acute liver failure.

As used herein, the term “non-alcoholic fatty liver disease” has its general meaning in the art and is intended to refer to the spectrum of disorders resulting from an accumulation of fat in liver cells in individuals with no history of excessive alcohol consumption. In the mildest form, NAFLD refers to hepatic steatosis. The term NAFLD is also intended to encompass the more severe and advanced form non-alcoholic steatohepatitis (NASH), cirrhosis, hepatocellular carcinoma, and virus-induced (e.g., HIV, hepatitis) fatty liver disease.

As used herein, the term “NASH” collectively refers to the state where the liver develops a hepatic disorder (e.g., inflammation, ballooning, fibrosis, cirrhosis, or cancer), or the state where the liver may induce such a pathological condition, and “NASH” is distinguished from “simple steatosis”; i.e., a condition in which fat is simply accumulated in the liver, and which does not progress to another hepatic-disorder-developing condition.

As used herein, the term “cirrhosis” refers to a consequence of chronic liver disease characterized by replacement of liver tissue by fibrosis, scar tissue and regenerative nodules (lumps that occur as a result of a process in which damaged tissue is regenerated), leading to loss of liver function. The term “alcohol-related cirrhosis” indicates that cirrhosis is mostly caused by excessive alcohol consumption.

As used herein, the term “etiology” refers to the causes or origins, of diseases or abnormal physiological conditions.

As used herein, the term “severity” refers to the degree of symptom intensity experienced, ascertained, formally assessed or reported by a symptomatic subject with a liver injury. Typically, the severity correlates with the Child-Pugh score.

As used herein, the term the “Child-Pugh score” has its general meaning in the art and refers to the score used to assess the prognosis of chronic liver disease, mainly cirrhosis as described by Child C G, Turcotte J G (1964). “Surgery and portal hypertension”. In Child C G (ed.). The liver and portal hypertension. Philadelphia: Saunders. pp. 50-64. Although it was originally used to predict mortality during surgery, it is now used to determine the prognosis, as well as the required strength of treatment and the necessity of liver transplantation. The score employs five clinical measures of liver disease including total bilirubin, serum albumin, prothrombin time prolongation (or INR), ascites and hepatic encephalopathy. Each measure is scored 1-3, with 3 indicating most severe derangement. Chronic liver disease is classified into Child-Pugh class A to C, as depicted in Table A.

TABLE A
Child-Pugh score and significance.

| | | One-year | Two-year |
|---|---|---|---|
| Points | Class | survival | survival |
| 5-6 | A | 100% | 85% |
| 7-9 | B | 80% | 60% |
| 10-15 | C | 45% | 35% |

As used herein, the term “albumin” has its general meaning in the art and refers to a globular protein that in humans is encoded by the ALB gene. Serum albumin is the most abundant plasma protein in mammals. Serum albumin is essential for maintaining the oncotic pressure needed for proper distribution of body fluids between intravascular compartments and body tissues. It also acts as a plasma carrier by non-specifically binding several hydrophobic steroid hormones and as a transport protein for hemin and fatty acids. Furthermore, serum albumin has a very long half-life of about 19 days, and its metabolism is well-known. Albumin has also been widely used as a protein stabilizer in commercial pharmaceuticals (Sangastino et al. (2012), Blood, 120(12) 2405-2411). An exemplary amino acid sequence for human serum albumin (HSA) is represented by SEQ D NO:1 (UniProtKB/Swiss-Prot primary accession number P02768).

>sp|P02768|ALBU_HUMAN Serum albumin OS = Homo
sapiens OX = 9606 GN = ALB PE = 1 SV = 2
SEQ ID NO: 1
MKWVTFISLLFLESSAYSRGVFRRDAHKSEVAHRFKDLGEENFKALVLIA
FAQYLQQCPFEDHVKLVNEVTEFAKTCVADESAENCDKSLHTLFGDKLCT
VATLRETYGEMADCCAKQEPERNECFLQHKDDNPNLPRLVRPEVDVMCTA
FHDNEETFLKKYLYEIARRHPYFYAPELLFFAKRYKAAFTECCQAADKAA
CLLPKLDELRDEGKASSAKQRLKCASLQKFGERAFKAWAVARLSQRFPKA
EFAEVSKLVTDLTKVHTECCHGDLLECADDRADLAKYICENQDSISSKLK
ECCEKPLLEKSHCIAEVENDEMPADLPSLAADFVESKDVCKNYAEAKDVF
LGMFLYEYARRHPDYSVVLLLRLAKTYETTLEKCCAAADPHECYAKVEDE
FKPLVEEPQNLIKQNCELFEQLGEYKFQNALLVRYTKKVPQVSTPTLVEV
SRNLGKVGSKCCKHPEAKRMPCAEDYLSVVLNQLCVLHEKTPVSDRVTKC
CTESLVNRRPCFSALEVDETYVPKEFNAETFTFHADICTLSEKERQIKKQ
TALVELVKHKPKATKEQLKAVMDDFAAFVEKCCKADDKETCFAEEGKKLV
AASQAALGL

As used herein, the term “isoform” has its general meaning in the art and refers to the multiple molecular forms of a given protein, and includes proteins differing at the level of (1) primary structure (such as due to alternate RNA splicing, or polymorphisms); (2) secondary structure (such as due to different co- or posttranslational modifications); and/or (3) tertiary or quaternary structure (such as due to different sub-unit interactions, homo- or hetero-oligomeric multimerization). According to the present, invention the term “isoform” preferably refers to the multiple molecular forms of a given protein, and includes proteins secondary structure due to different co- or post translational modifications. Said post translational modifications include cysteinylation, homocysteinylation, glutathionylation, glycation, nitrosylation, nitration, oxidation and carbonylation.

As used herein, the term “native albumin” refers to the form of albumin that was not subjected to a modification, more particularly a post translational modification.

As used herein, the term “Alb-DA” refers to an albumin isoform characterized by a truncation of the N-terminus end.

As used herein, the term “Alb-DA+Cys” refers to an albumin isoform characterized by a truncation of the N-terminus end and a cysteinylation of the Cys34.

As used herein, the term “Alb+SO2H” refers to an albumin isoform characterized a deoxidation.

As used herein, the term “Alb+SO3H” or “Alb-CysO3” refers to an albumin isoform characterized by a trioxidation.

As used herein, the term “Alb+Cys-DHA” refers an albumin isoform characterized by a cysteinylation of the Cys34 and a transformation of a free cysteine to a dehydroalanine.

As used herein, the term “Alb+Cys+SNO” refers to an albumin isoform characterized by a cysteinylation of the Cys34 and a nitrosylation.

As used herein, the term “Alb+Glyc” refers to an albumin isoform characterized by a glycation/As used herein, the term “Alb+SO2H+Glyc” refers to an albumin isoform characterized by a dioxidation and a glycation.

As used herein, the term “Alb+SO3H+Glyc” refers to an albumin isoform characterized by a trioxidation and a glycation.

As used herein, the term “Alb+Cys+Glyc-DHA” refer to an albumin isoform characterized by a cysteinylation of the Cys34, a glycation and a transformation of a free cysteine to a dehydroalanine.

As used herein, the term “Alb+Cys+Glyc” refers to an albumin isoform characterized by a cysteinylation of the Cys34 and a glycation.

As used herein, the term “Alb-SGGS” refers to an albumin isoform characterized by a glutathionylation.

As used herein, the term “Alb+2Glyc” refers to an albumin isoform characterized by two glycations.

As used herein, the term “Alb+SO3H+2Glyc” refers to an albumin isoform characterized by a trioxidation and two glycations.

As used herein, the term “Alb+Cys+2Glyc” refers to an albumin isoform characterized by a cysteinylation of the Cys34 and two glycations.

As used herein, the term “profile” means a pattern and relates to the magnitude and direction of change of a number of features. The profile may be interpreted stringently, i.e., where the variation in the magnitude and/or number of features within the profile displaying the characteristic is substantially similar to a reference profile or it may be interpreted less stringently, for example, by requiring a trend rather than an absolute match of all or a subset of feature characteristics.

As used herein, the term “mass spectrometry” or “MS” refers to an analytical technique to identify compounds by their mass. MS refers to methods of filtering, detecting, and measuring ions based on their m/z. MS technology generally includes (1) ionizing the compounds to form charged species (e.g., ions); and (2) detecting the molecular weight of the ions and calculating their m/z. The compounds may be ionized and detected by any suitable means. A “mass spectrometer” generally includes an ionizer and an ion detector. In general, one or more molecules of interest are ionized, and the ions are subsequently introduced into a mass spectrometric instrument where, due to a combination of magnetic and electric fields, the ions follow a path in space that is dependent upon mass (“m”) and charge (“z”). See, e.g., U.S. Pat. No. 6,204,500, entitled “Mass Spectrometry From Surfaces;” U.S. Pat. No. 6,107,623, entitled “Methods and Apparatus for Tandem Mass Spectrometry;” U.S. Pat. No. 6,268,144, entitled “DNA Diagnostics Based On Mass Spectrometry;” U.S. Pat. No. 6,124,137, entitled “Surface-Enhanced Photolabile Attachment And Release For Desorption And Detection Of Analytes;” Wright et al., Prostate Cancer and Prostatic Diseases 2:264-76 (1999); and Merchant and Weinberger, Electrophoresis 21:1164-67 (2000).

As used herein, the term “blood sample” means a whole blood, serum, or plasma sample obtained from the patient or the animal.

### Methods of the Present Invention

The first object of the present invention relates to a method of determining the etiology and severity of a liver injury in a subject comprising determining the profile of albumin isoforms in a blood sample obtained from the subject wherein the profile indicates the etiology and severity of the liver injury.

More particularly, the method of the present invention comprises the step of detecting a plurality of albumin isoforms. Even, more particularly, the method of the present invention comprises the step of detecting a plurality of albumin isoforms selected from Table 4. In some embodiments, the method of the present invention comprises the step of detecting a plurality of isoforms selected from the group consisting of Alb+SO2H, HSA-CysO3, Alb+Cys-DHA, Alb+Cys, Alb+Cys+SNO, Alb+SO2H+Glyc, Alb+SO3H+Glyc, HAS-SGGS, Alb+2Glyc, Alb+SO3H+2Glyc, and Alb+Cys+2Glyc.

In some embodiments, the method of the present invention comprises the steps of i) determining the profile of albumin isoforms in the blood sample obtained from the patient, and ii) comparing the profile to one or more reference profiles associated with various liver injuries.

The method of the present invention is particularly suitable for detecting any kind of liver injury. In some embodiments, the method of the present invention is particularly suitable for detecting chemical liver injuries. In some embodiments, the method of the present invention is particularly suitable for detecting physical liver injuries. In some embodiments, the method of the present invention is particularly suitable for detecting ischemic liver injuries.

The method of the present invention is particularly suitable for the early detection of a liver injury, i.e. the detection of a liver injury before the observation of a symptom.

The method of the present invention is particularly suitable for the early detection of early graft dysfunction or non-function in liver transplanted patients.

In particular, the method of the present invention is particularly suitable for detecting a chemical liver injury induced by a toxicant selected from the group consisting of alcohol, 2,2′,4,4′,5,5′-hexachlorobiphenyl (PCB-153), 2,3,7,8-tetrachlorodibenzo-p-dioxin (TCDD), 2-bromoethylamine (BEA), 3-methylcholanthrene, 4-aminophenol (PAP), acetaminophen (APAP), adriamycin, allyl alcohol, amiodarone, amphotericin B, Aroclor 1254, Aroclor 1260, arsenic, aspirin, astemizole, benzene, cadmium, carbamezipine, carbon tetrachloride (CCl4), ciprofibrate (cipro), clofibrate, cobalt chloride, corvastatin, cyclosporin A, diethylnitrosamine, dimethylformamide, dimethylhydrazine (DMH), diquat, ethosuximide, etoposide, famotidine, fluconazole, gemfibrozil, ganciclovir, hexachloro-1,3-butadiene (HCBD), HIV protease inhibitors, hydrazine, indomethacin, ketoconazole, lead acetate (PbAc), lipopolysaccharide (LPS), mercury(II) chloride (HgCl₂), methanol, methapyrilene, methotrexate, metronidazole, miconazole, monocrotaline, nitric oxide, ondansetron, pentamidine, phenobarbital, phenylhydrazine (phenylhyrzn), phenytoin, pravastatin, propulsid, puromycin aminonucleoside (PAN), quinolones, simvastatin, sodium fluoride (NaF), statins, thioacetamide, tocainidine, tricyclic antidepressants, troglitazone, tumor necrosis factor α (TNFα), uranyl nitrate, valproic acid, vincristine, Wy-16,463, zidovudine (AZT), α-naphthyl isothiocyanate (ANIT), β-naphthoflavone (BNF), asbestos, radon, cigarette smoke, glues, dioxin, nickel, arsenic, mercury, cement (chromium), polychlorinated biphenyls (PCBs), carbon tetrachloride, methylene chloride, vinyl chloride, mercury, chlorinated hydrocarbon solvents, carbon disulfide, cadmium, ozone, tobacco smoke, nitrates, methylene chloride, ethylene dibromide, and polychlorinated biphenyls.

The method of the present invention is particularly suitable for detecting a liver injury selected from the group consisting of liver abscess, liver cancer, either primary or metastatic, cirrhosis, such as cirrhosis caused by the alcohol consumption or primary biliary cirrhosis, amebic liver abscess, autoimmune hepatitis, biliary atresia, coccidioidomycosis disseminated, portal hypertension hepatic infections (such as hepatitis A virus, hepatitis B virus, hepatitis C virus, hepatitis D virus, or hepatitis E virus), hemochromatosis, hepatocellular carcinoma, pyogenic liver abscess, Reye's syndrome, sclerosing cholangitis, Wilson's disease, drug induced hepatotoxicity, or fulminant or acute liver failure.

The method of the present invention is particularly suitable for detecting a non-alcoholic fatty liver disease, and in particular for detecting NASH.

The method of the present invention is particularly suitable for detecting liver fibrosis. The method of the present invention is particularly suitable for detecting cirrhosis.

In some embodiments, the method of diagnosing described herein is applied to a subject who presents symptoms of liver injury without having undergone the routine screening to rule out all possible causes for liver injury. The methods described herein can be part of the routine set of tests performed on a subject who presents symptoms of liver injury such as jaundice, abdominal pain and swelling, swelling in the legs and ankles, itchy skin, dark urine color, pale stool color, bloody color stool, tar-colored stool, chronic fatigue, nausea or vomiting, loss of appetite, tendency to bruise easily . . . . The method of the present invention can be carried out in addition of other diagnostic tools that include ultrasound evaluation (e.g. elastography), biopsy and/or quantification of at least one further biomarkers such as levels of blood AST, ALT, ALP, TTT, ZTT, total bilirubin, total protein, albumin, lactate dehydrogenase, choline esterase and the like.

A further object to the present invention relates to a method of predicting the worsening of a liver injury comprising the steps of determining the evolution of the profile of albumin isoforms in the blood sample obtained from the patient wherein said evolution predicts the worsening of the liver injury.

A further object of the present invention relates to a method of predicting an early allograft liver dysfunction or liver non-function in a liver-transplanted patient comprising the steps of determining the evolution of the profile of albumin isoforms in the blood sample obtained from the patient wherein said evolution predicts the early allograft dysfunction or non-function.

In some embodiments, the isoforms are detected by mass spectrometry. In some embodiments, the isoform is identified visually by its mass shift with respect to native albumin and by taking the maximum intensity in a mass interval of 10 Da around the observed peak of each isoform. Table 4 indicates the different mass shifts that are associated with Alb+SO2H, HSA-CysO3, Alb+Cys-DHA, Alb+Cys, Alb+Cys+SNO, Alb+SO2H+Glyc, Alb+SO3H+Glyc, HSA-SGGS, Alb+2Glyc, Alb+SO3H+2Glyc, and Alb+Cys+2Glyc.

Mass spectrometry is performed using a mass spectrometer, which includes an ion source for ionizing the fractionated sample and creating charged molecules for further analysis. Ionization sources used in various MS techniques include, but are not limited to, electron ionization, chemical ionization, electrospray ionization (ESI), photon ionization, atmospheric pressure chemical ionization (APCI), photoionization, atmospheric pressure photoionization (APPI), fast atom bombardment (FAB)/liquid secondary ionization (LSIMS), matrix assisted laser desorption ionization (MALDI), field ionization, field desorption, thermospray/plasmaspray ionization, surface enhanced laser desorption ionization (SELDI), inductively coupled plasma (ICP) and particle beam ionization. The skilled artisan will understand that the choice of ionization method may be determined based on the analyte to be measured, type of sample, the type of detector, the choice of positive versus negative mode, etc. After the sample has been ionized, the positively charged ions thereby created may be analyzed to determine m/z. Suitable analyzers for determining m/z include quadrupole analyzers, ion trap analyzers, and time-of-flight analyzers. The ions may be detected using one of several detection modes. For example, only selected ions may be detected using a selective ion monitoring mode (SIM), or alternatively, multiple ions may be detected using a scanning mode, e.g., multiple reaction monitoring (MRM) or selected reaction monitoring (SRM). One may enhance the resolution of the MS technique by employing “tandem mass spectrometry,” or “MS/MS.” In this technique, a precursor ion (also called a parent ion) generated from a molecule of interest can be filtered in an MS instrument, and the precursor ion subsequently fragmented to yield one or more fragment ions (also called daughter ions or product ions) that are then analyzed in a second MS procedure. By careful selection of precursor ions, only ions produced by certain analytes are passed to the fragmentation chamber, where collision with atoms of an inert gas produce the fragment ions. Because both the precursor and fragment ions are produced in a reproducible fashion under a given set of ionization/fragmentation conditions, the MS/MS technique may provide an extremely powerful analytical tool. For example, the combination of filtration/fragmentation may be used to eliminate interfering substances, and may be particularly useful in complex samples, such as biological samples. Additionally, recent advances in technology, such as matrix-assisted laser desorption ionization coupled with time-of-flight analyzers (“MALDI-TOF”) permit the analysis of analytes at femtomole levels in very short ion pulses. Mass spectrometers that combine time-of-flight analyzers with tandem MS are also well known to the artisan. Additionally, multiple mass spectrometry steps may be combined in methods known as “MS/MS”. Various other combinations may be employed, such as MS/MS/TOF, MALDI/MS/MS/TOF, or SELDI/MS/MS/TOF mass spectrometry.

Typically the blood samples are processed to obtain preparations that are suitable for analysis by mass spectrometry. Such purification will usually include chromatography, such as liquid chromatography or capillary electrophoresis, and may also often involve an additional purification procedure that is performed prior to chromatography. Various procedures may be used for this purpose depending on the type of sample or the type of chromatography. Examples include filtration, centrifugation, combinations thereof and the like. The pH of the serum sample may then be adjusted. The sample may be purified with a filtration. The filtrate from this filtration can then be purified by liquid chromatography and subsequently subjected to mass spectrometry analysis. Various methods have been described involving the use of high performance liquid chromatography (HPLC) for sample clean-up prior to mass spectrometry analysis. See, e.g., Taylor et al., Therapeutic Drug Monitoring 22:608-12 (2000) (manual precipitation of blood samples, followed by manual C18 solid phase extraction, injection into an IPLC for chromatography on a C18 analytical column, and MS/MS analysis); and Salm et al., Clin. Therapeutics 22 Supl. B:B71-B85 (2000). Commercially available IPLC columns include, but are not limited to, polar, ion exchange (both cation and anion), hydrophobic interaction, phenyl, C-2, C-8, C-18, and polar coating on porous polymer columns. During chromatography, the separation of materials is effected by variables such as choice of eluent (also known as a “mobile phase”), choice of gradient elution and the gradient conditions, temperature, etc.

One or more steps of the methods may be performed using automated machines. In some embodiments, one or more purification steps are performed on-line, and more preferably all of the LC purification and mass spectrometry steps may be performed in an on-line fashion.

In some embodiments, the method of the invention comprises the use of an algorithm. More particularly, the method of the present invention is a computer-implemented method comprising applying, on a set of values for each detected isoforms relative to the subject, a trained model configured to determine the etiology and severity of the liver injury based on the set of values. Typically, the set of values comprises the relative abundance of the different detected isoforms. In some embodiments, the model is preliminary trained by supervised learning on a training dataset comprising, for a plurality of individuals of a population, the etiologies and the severities of a liver injury. In some embodiments, the method of the invention thus comprises the use of a classification algorithm typically selected from Linear Discriminant Analysis (LDA), Topological Data Analysis (TDA), Neural Networks, Support Vector Machine (SVM) algorithm and Random Forests algorithm (RF). As used herein, the term “classification algorithm” has its general meaning in the art and refers to classification and regression tree methods and multivariate classification well known in the art such as described in U.S. Pat. No. 8,126,690; WO2008/156617. Thus, in some embodiments, the method of the present invention comprises a) detecting the plurality of albumin isoforms; b) implementing a classification algorithm on data relative to the detected isoforms so as to obtain an algorithm output; c) determining the liver injury and severity. The algorithm of the present invention can be performed by one or more programmable processors executing one or more computer programs to perform functions by operating on input data and generating output. The algorithm can also be performed by, and apparatus can also be implemented as, special purpose logic circuitry, e.g., an FPGA (field programmable gate array) or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. In some embodiments, the algorithm can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components.

The result given by the methods of the invention may be used as a guide in selecting a therapy or treatment regimen for the subject. Typically, the patient can be then eligible for intensive surveillance (e.g., referral to tertiary care centers; intensive control of risk factors), for a selected therapy or transplantation and for inclusion in clinical trials testing new drugs. According to the present invention, the treatment consists in any method or drug that could be suitable for the treatment of a liver injury. Some liver problems can be treated with lifestyle modifications, such as stopping alcohol use or losing weight, typically as part of a medical program that includes careful monitoring of liver function. Each liver disease will have its own specific treatment regimen. For example, hepatitis A requires supportive care to maintain hydration while the body's immune system fights and resolves the infection. Patients with gallstones may require surgery to remove the gallbladder. Other diseases may need long-term medical care to control and minimize the consequences of their disease. In patients with cirrhosis and end-stage liver disease, medications may be required to control the amount of protein absorbed in the diet. Other examples include operations required to treat portal hypertension. The patient can also be eligible for administration of corticosteroids, pentoxifylline, or N-acetylcysteine; antiapoptotics, or vasoactive drugs) or even for liver transplantation.

The method of the present invention can also be useful for monitoring the subject. In some embodiments, the method of the present invention is also particularly suitable for determining whether a subject suffering from a liver injury achieves a response to a therapy. The method is thus particularly suitable for discriminating responder from non-responder. As used herein the term “responder” in the context of the present disclosure refers to a subject that will achieve a response, i.e. a subject who is under remission and more particularly a subject who does not suffer from liver injury. A “non-responder” subject includes subjects for whom the disease does not show reduction or improvement after the treatment (e.g. the liver injury remains stable or decreases). For instance, the nature and the abundance of the different isoforms can indeed be monitored during the treatment of the subject and thus can indicate whether the subject achieves a response to the therapy.

The invention will be further illustrated by the following figures and examples. However, these examples and figures should not be interpreted in any way as limiting the scope of the present invention.

### FIGURES

FIG. 1. Evolution of albumin isoforms in the different groups of rats exposed to ethanol for different time durations. Native albumin is expressed as percent of the sum of all detected isoforms. Other isoforms are expressed in relative abundance to that of native albumin.

FIG. 2. Evolution of albumin isoforms in the different groups of rats exposed to CCl₄ for different time durations. Native albumin is expressed as percent of the sum of all detected isoforms. Other isoforms are expressed in relative abundances of the native albumin.

FIG. 3. Evolution of albumin isoforms in the different groups of rats exposed to APAP at different doses and for different durations. Native albumin is expressed as percent of the sum of all detected isoforms. Other isoforms are expressed in relative abundances of the native albumin.

FIG. 4: A. median albumin profiles of rats intoxicated with CCl₄; B. median albumin profiles of rats intoxicated with Ethanol, C. median profiles of rats intoxicated with APAP. The different lines represent the control group for each model; the profiles of groups in which biochemistry tests started to be altered and the groups where albumin profiles were the most disturbed.

FIG. 5. PCA-DA plot of the 3 animal models. Each dot represents the whole information of the albumin isoform profile for one rat. The different dots represent rats exposed to ethanol, rats exposed to APAP and are rats exposed to CCl₄.

FIG. 6. Histograms of albumin isoforms in cirrhotic patients.

FIG. 7. A. PCA-DA plot of the 4 groups of patients. Each dot represents the whole information of the albumin profile for one patient. Square dots are control patients, orange dots represent patients with ALD, circle dots are NASH patients and triangle dots patients with cirrhosis of mixed origin. B. PCA-DA plot of 3 groups of patients: Controls, NASH and mixed origin.

FIG. 8. A. PCA-DA plot of the 4 groups of patients. Each dot represents the whole information of the albumin profile for one patient. Square dots are control patients, circle dots represent stage A, circles dots stage B, triangle dots represent stage C cirrhosis patients. B. PCA-DA plot of 3 groups of patients: stage A, B and C.

FIG. 9. PLS-DA plot of 2 groups of patients. EAD+ are patients in whom early allograft dysfunction has been diagnosed based on the LGrAF T10 or the EAD scores. EAD− are patients with no graft dysfunction detected. Each dot represents the whole information of the albumin profile for one patient at V1 (24 hours before transplantation) subtracted from the whole information of the albumin profile at V6 (7 days after transplantation).

FIG. 10. PLS-DA plot of 2 groups of patients. EAD+ are patients in whom early allograft dysfunction has been diagnosed based on the LGrAFT10 or the EAD scores. EAD− are patients with no graft dysfunction detected. Each dot represents the whole information of the albumin profile for one patient at V1 (24 hours before transplantation) subtracted from the whole information of the albumin profile at V3 (72 hours after transplantation).

### EXAMPLE

### Methods

### Materials and Methods

### 1. Animals

All of the experimental procedures were performed in male, adult (6 weeks old) albino Wistar rats (Janvier Labs, France) weighing an average of 250 grams and maintained under normal temperature (21 °C) and humidity and a 12-hour light-dark cycle with unrestricted access to food and water. Rats were acclimatized to the conditions of our animal facility for 1 week before the start of the investigations.

All animal experiments had been approved by the Ethics Committee for experiments at the University of Limoges and by the French Ministry of National Education, Higher Education and Research (APAFiS #20354-2019042414581742 v1).

### 2. Experimental Procedure

### 2.1. Model of Ethanol (EtOH) Hepatotoxicity:

In the EtOH model, hepatotoxicity was induced by oral administration (gavage) of 2 mL of a 50% EtOH solution prepared in physiological saline (0.9% NaCl) to evaluate the time-dependent changes in biochemical markers and histological liver injuries. Six different groups of 6 to 9 rats were followed for 1, 3, 7, 10, or 14 days, respectively. All rats received the daily dose of EtOH. The animals were sacrificed 24 hours after their last intake of EtOH. “Control” rats were followed throughout the duration of the protocol, ie 14 days, and received by gavage only physiological serum (0.9% NaCl).

### 2.2. Model of Carbon Tetrachloride (CCl₄) Hepatotoxicity:

In the CCl₄ model, hepatotoxicity was induced by oral administration (gavage) of 1 mL/kg body weight of a 30% solution of CCl₄ diluted in olive oil. Five groups of rats were followed for 1, 3, 7, 10 days after daily administration of CCl₄, respectively. The animals were sacrificed 24 hours after their last intake of CCl₄. “Control” rats were followed for 10 days and received by gavage only olive oil.

### 2.3. Model of Paracetamol (APAP) Hepatotoxicity:

In the APAP model, rats were randomly divided into eleven groups, each group containing six rats. Depending on the group, the animals received APAP suspensions prepared in 1% carboxymethylcellulose at doses D1=1 g/kg, D2=2 g/kg, D3=3 g/kg or D4=4 g/kg. APAP was administrated for either 1 day or 3 days. “Control” rats were followed for 3 days and received by gavage only a single dose of 1% carboxymethylcellulose. The animals were sacrificed 24 h after the last dose of APAP.

During the experimental period, body weight was recorded daily until sacrifice. For each experimental model, the animals were sacrificed by intraperitoneal injection of pentobarbital (150 mg/kg).

Blood samples were collected in Vacutainer® lithium heparin tube for trace elements (Beckton Dickinson, France) and then centrifuged at 3000 rpm for 10 minutes. The plasma samples were then stored at −80 °C until analysis. Rat livers were quickly removed and fixed in formalin for histological analysis.

### 3. Analytical Procedures

### 3.1. Biochemistry Analyzes

From the collected plasma, measurements of classic biochemistry parameters such as albumin (ALB), total (BILIT) and conjugated (BILID) bilirubin, aspartate aminotransferases (ASAT), alanine aminotransferases (ALAT) and alkaline phosphatases (PAL) were evaluated in the Biochemistry and Molecular Genetics laboratory of Limoges University Hospital using a COBAS® 8000 automaton (Roche, Germany).

### 3.2. Pathological Analysis of the Liver

At the end of each sacrifice, the livers were cut into sections of 1 to 1.5 cm perpendicular to the major axis to allow homogeneous fixation in a 4% formalin solution for a maximum of 7 days. Samples were stained for light microscopy with hematoxylin and eosin staining and Masson's trichrome stain. The pathologist performed histological analysis blindly, with no knowledge of the different experimental groups.

### 3.3. Proteomic Analysis of the Plasma Samples (Identification-Quantification of the Different Isoforms of Albumin)

### Standards and Reagents

Organic solvents and reagents were of analytical grade. Acetonitrile was obtained from Merck (Molsheim, France), ammonium formate and formic acid from Sigma (Saint-Quentin-Fallavier, France). Deionized water was prepared on a Direct-Q laboratory plant (Millipore, Molsheim, France).

### Sample Preparation

A volume of 20 μL of plasma was diluted with 980 μL of an aqueous 20 mM ammonium formate solution with 0.1% formic acid, and then vortex-mixed. The mixture was centrifuged at 10 °C and 14 000 g, and 300 μl of the supernatant was then filtered on a 0.22 μm cellulose acetate filter before injection.

### Liquid Chromatography—Mass Spectrometry

Chromatographic separation was performed using a Nexera LC40 system (Shimadzu, Noisiel, France) equipped with a thermostated column compartment and a thermostated autosampler with a six-port switching valve. Samples were analyzed without chromatographic separation under isocratic conditions using a 2 mM aqueous ammonium formate solution containing 0.1% formic acid as mobile phase A and a mixture of acetonitrile/mobile phase A (90:10, by volume) as mobile phase B, programmed as follows: 0-3 min, 50% B.

Mass spectrometric detection was performed using a Q-TOF mass spectrometer (TripleTOF® 5600+, Sciex, Concord, Canada) equipped with a DuoSpray ion source and operated in the positive ionization mode. A beta-galactosidase solution was used for internal calibration. The source conditions were as follows: temperature, 200 °C; declustering potential (DP), 250 eV; curtain gas (CUR), 40 units; ion source gas (GS1, GS2), respectively 70 and 10 units; and ion-spray voltage floating, 5.5 kV. All MS parameters were controlled by Analyst® TF 1.7 (Sciex). Data were processed with PeakView® 2.2 software (Sciex). m/z ratios were scanned using a TOF MS scan from m/z 900 and 1800 with an accumulation time of 500 ms.

### Spectra Deconvolution

The LC-MS data were processed using PeakView® 2.2 software and its Bio Tool Kit 2.2.0 feature (Sciex). The input MS spectra selected from 1300 to 1600 was then deconvolved using a low resolution (5000) from m/z 1000 to 200000. Deconvolution spectra (or profiles) are expressed as intensity versus mass in Da.

### Estimation of the Relative Abundance of Isoforms.

The relative abundance of each isoform was determined after calculating the ratio between each centroided peak and the total area represented by the sum of the centroid peaks between 65500 and 67000 for rat serum albumin or 66000 to 67500 for human serum albumin.

Identification and quantification (intensity and relative abundance) of isoforms Each isoform was identified visually by its mass shift with respect to native albumin and by taking the maximum intensity in a mass interval of 10 Da around the observed peak of each isoform.

The relative proportion of the intensity of each isoform was calculated by dividing the intensity or area obtained from its deconvoluted spectrum by the summed intensity of all isoforms (between 65500 and 67000) and multiplying it by 100. Data were gathered in an Excel file then analyzed using GraphPad® for the potential isoforms.

Relative abundance was calculated as the ratio between the maximum intensity of each identified isoform compared that of the native isoform.

### 4. Patients and Samples

Patient plasma samples were all leftovers after biochemistry laboratory tests prescribed according to the standard of care. In accordance with French regulations and Good Clinical Practice for biomedical studies, patients were informed of, and were able to oppose to, the use of the leftovers of their blood samples at any time (CSP article L1211-2).

The cohort was composed of cirrhotic patients and of patients with no liver dysfunction as controls. Patients were considered as free from liver dysfunction on the basis of their clinical diagnosis and their liver function biochemical tests, namely, aspartate transaminase, alanine transaminase, alkaline phosphatase, x-glutamyltransferase, free and total bilirubin, and albumin levels.

Cirrhotic patients were included based on an hepatologist's diagnosis, their liver function biochemical tests and their Child-Pugh scores.

Patient plasma samples were prepared as previously described and albumin isoforms were analyzed and identified as previously described for rat plasma samples.

### 5. Statistical Analyses

The results of the biochemical analyses are presented as the median [min−max].

Statistical probabilities of p<0.01 (*) and p<0.05 (**) were considered significant. All analyses and figures were performed using GraphPad Prism 7.0a software (GraphPad Software, San Diego, USA).

### Results

### Precocity of Albumin Modifications

### SEB Test in Acute Models (EtOH and CCl₄):

In rats exposed to EtOH and CCl₄, hepatic cytolysis was observed starting at D7. In the EtOH model, it was further increased, together with a significant increase in ALT levels, on D10 and D14. In this model, a significant increase in AST level was observed at D7, D10 and D14 (Table 1). In the CCl₄ model, a significant increase in AST and ALT levels at all time points and a significant increase in bilirubin (BILIT & BILID) at D7 and D10 were detected (Table 2), suggesting hepatic cytolysis and cholestasis. Histologically, only minor inflammation was detected in some of the rats receiving ethanol. Rats exposed to CCl₄ showed steatosis starting at D3 and only 2 rats had a fibrotic liver tissue (1 in the D3 group and 1 in the D10 group).

### SEB Test and Albumin Isoforms in the Acute Hepatotoxicity Models:

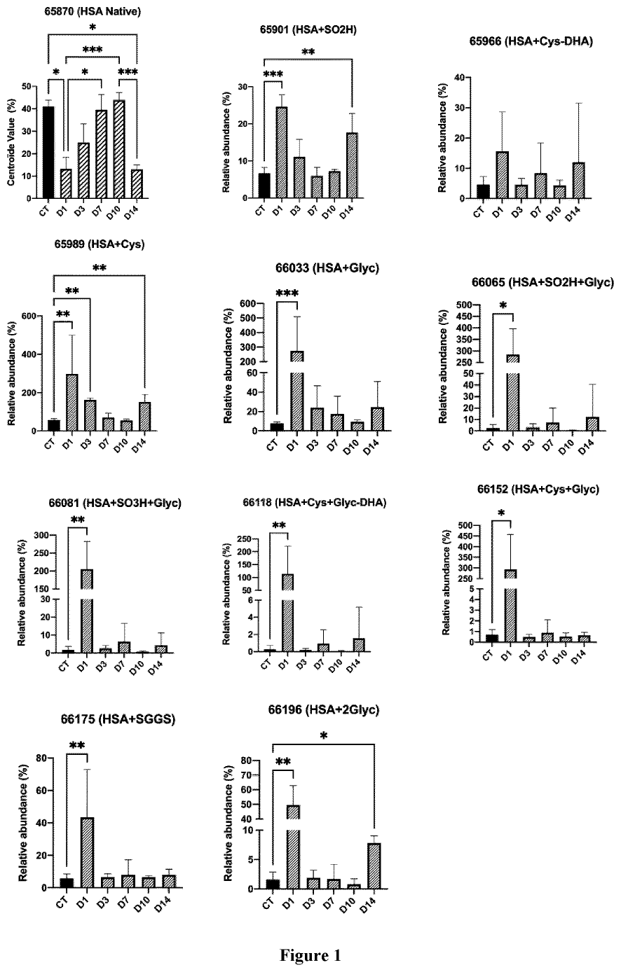

Most ligands of the SEB test increased starting D1 in both models except for Cd that increased only in EtOH model at D14 (FIG. 1). A normalization of Au, L-Thyroxine was observed at D7 in both models followed by secondary increase at D10 or D14 for Au and dansylsarcosine.

In the EtOH model, In addition to the native albumin with a mass of 65870, 12 potential isoforms were identified, namely Alb+SO2H, Alb-CysO3, Alb+Cys-DHA, Alb+Cys, Alb+Cys+SNO, Alb+Glyc, Alb+SO2H+Glyc, Alb+SO3H+Glyc, Alb+Cys+Glyc+DHA, Alb+Cys+Glyc, Alb+2Glyc and, Alb+Cys+2Glyc. All isoforms except Alb+CysO₃ and Alb+Cys+Glyc were also detected in the CCl₄ model, in addition to another isoform, Alb+SO3H+2Glyc (isoforms and corresponding mass shifts are listed in Table 4).

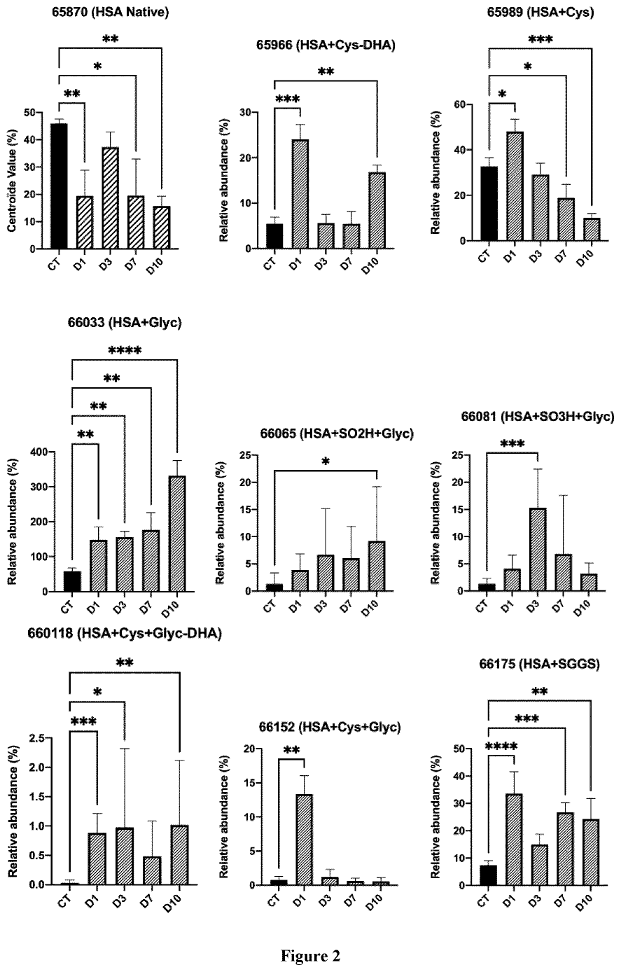

In both models, native albumin decreased rapidly after the administration of the hepatotoxic compound, as soon as D1 for some. It increased again at D3 in the CCl₄ model and at D7 in the EtOH model then dropped again. These modifications were paralleled by the increase of several isoforms as shown in FIGS. 1 and 2.

### Gradual Acute Model (APAP):

In rats acutely intoxicated with APAP for 2 (D2), 3 (D3) or 4 (D4) days (with doses of 2, 3 and 4 g/kg per day, respectively), classical biochemistry markers were not different from those in the control groups (Table 3) except a transient increase of AST in the D2g-D1 group due to an outlier and a slight but significant decrease of total albumin. Histological examination of liver tissues revealed inflammation in some rats, mostly those sacrificed 72 h after receiving 4 g/kg of APAP (D4g-D3). Necrosis was observed only in the group D4g-D1 (5 rats out of 6) but was not visible in the group D4g-D3.

### Albumin Isoforms Analysis of APAP Exposed Rats:

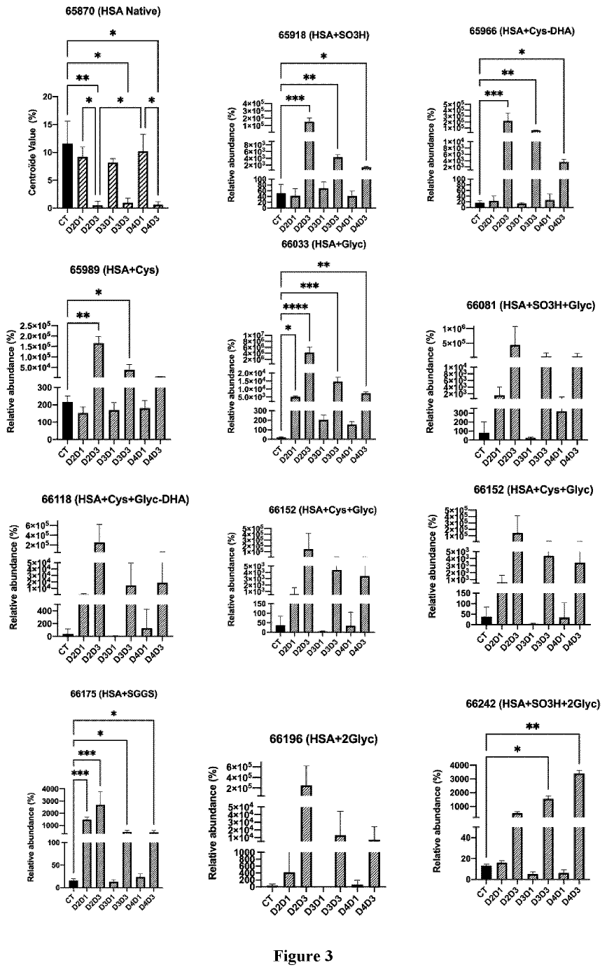

In addition to native albumin, we have identified 11 isoforms in the serum of rats exposed to APAP, namely, Alb+SO₂H, HSA-CysO₃, Alb+Cys-DHA, Alb+Cys, Alb+Cys+SNO, Alb+SO₂H+Glyc, Alb+SO₃H+Glyc, HSA+SGGS, Alb+2Glyc, Alb+SO₃H+2Glyc, Alb+Cys+2Glyc. All the isoforms were also present in the control group. However, native albumin decreased dramatically in all groups in respect to control group, particularly in the groups sacrificed 3 days after APAP initiation, namely, groups D2gD3, D3gD3 and D4gD3 (FIG. 3). Albumin in all rats was intensely modified. The percentage of native albumin gradually dropped from 12% in the control group to less than 1% in the groups sacrificed at D3. In parallel, several isoforms increased dramatically, up to 10³-fold, in some groups (FIG. 3). This is principally due to the huge decrease of native albumin in this model. For example, Alb+Cys intensities were 2-fold higher than native Alb in the control group; after 3 days of daily APAP administration, this isoform increased to more than 200-times the concentration of native Alb.

### Albumin Isoform Profiles as a Signature of the Nature and the Intensity of Liver Injuries

### Animal Experiments:

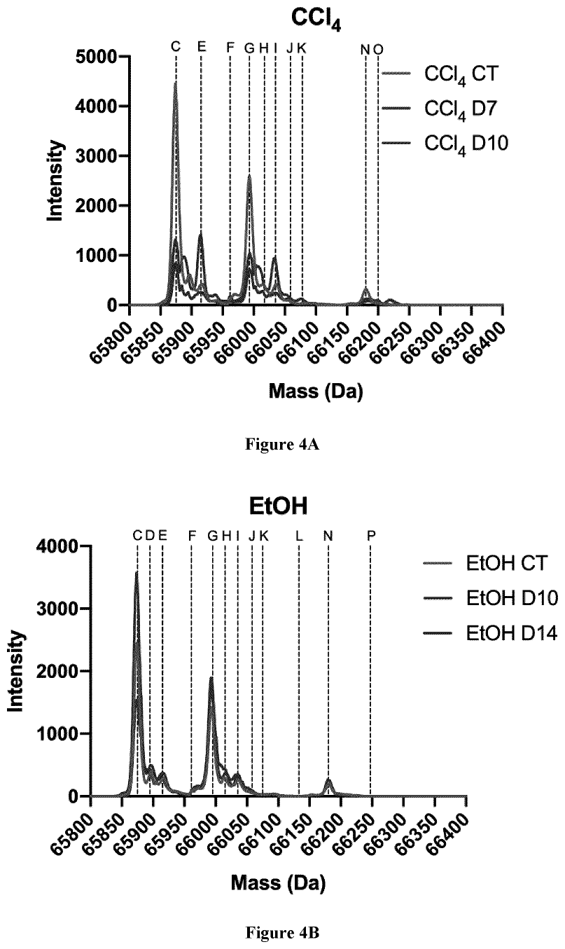

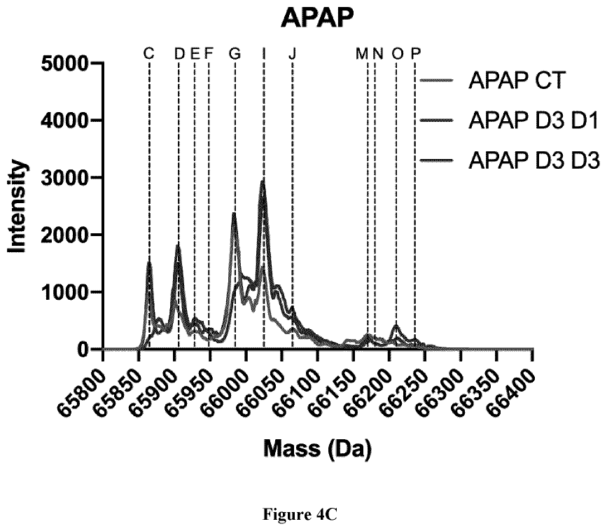

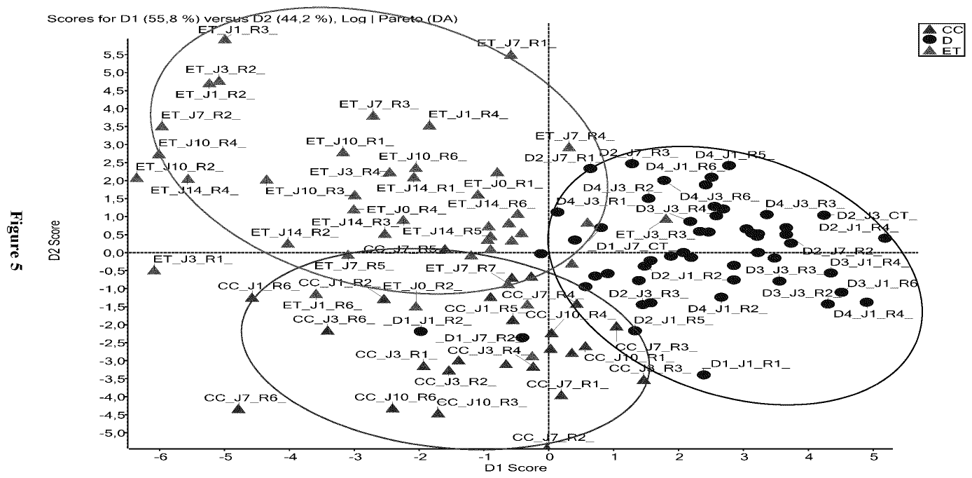

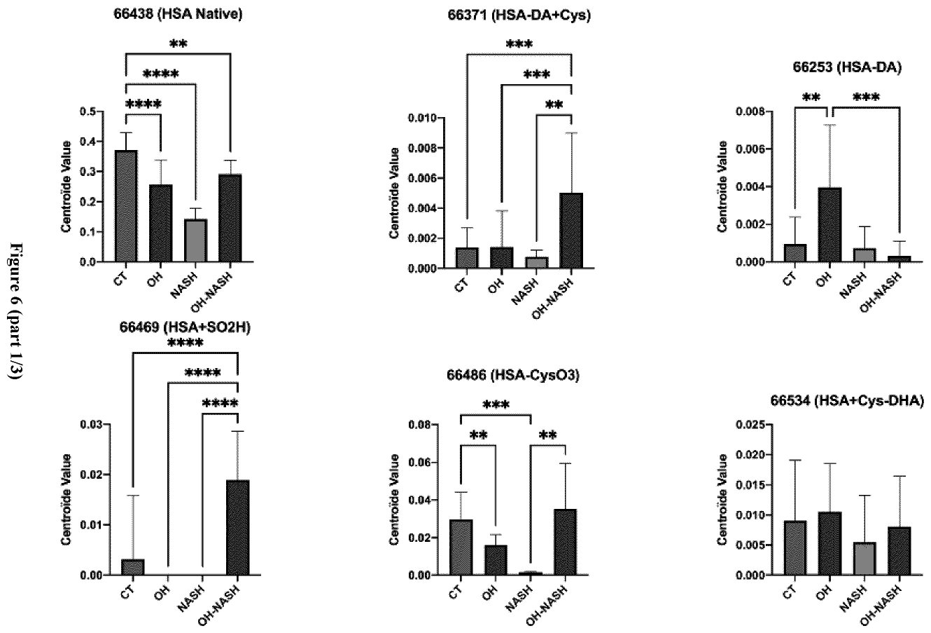

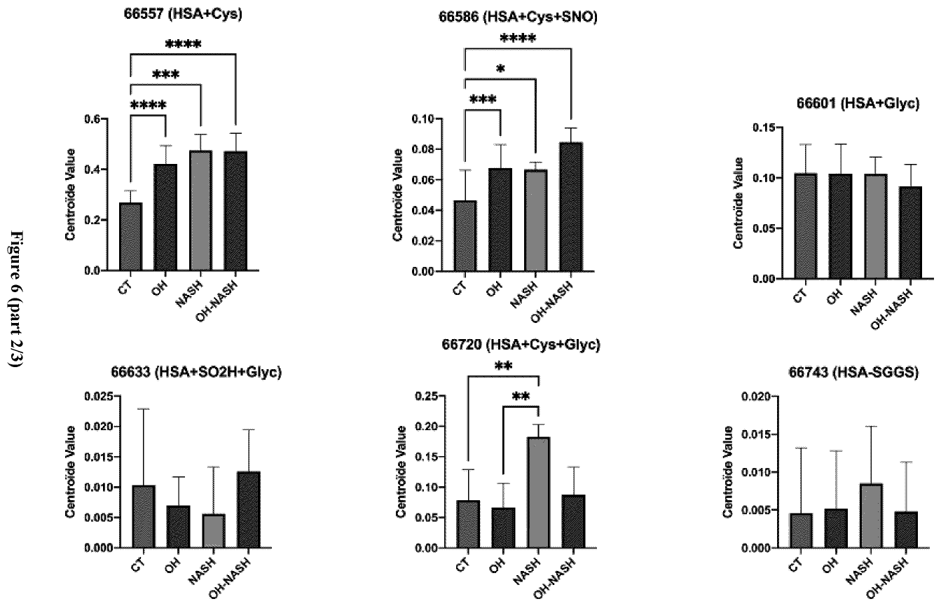

As shown in the previous paragraphs, the decrease of native albumin concentration was associated with the increase of certain isoforms, varying with the toxicant (Table 4) and resulting in profiles of albumin isoforms that were visually different (FIG. 4). Therefore, we then considered the whole MS profile for each model. A supervised principal component analysis (PCA-DA) revealed a clear clustering of the 3 different groups (FIG. 5).

### Albumin Isoform Profiles in Cirrhotic Patients:

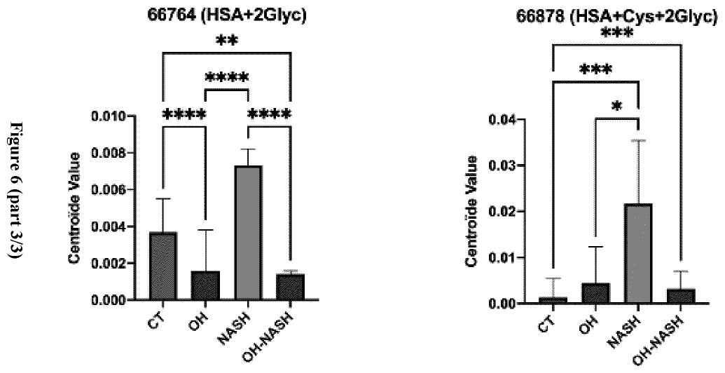

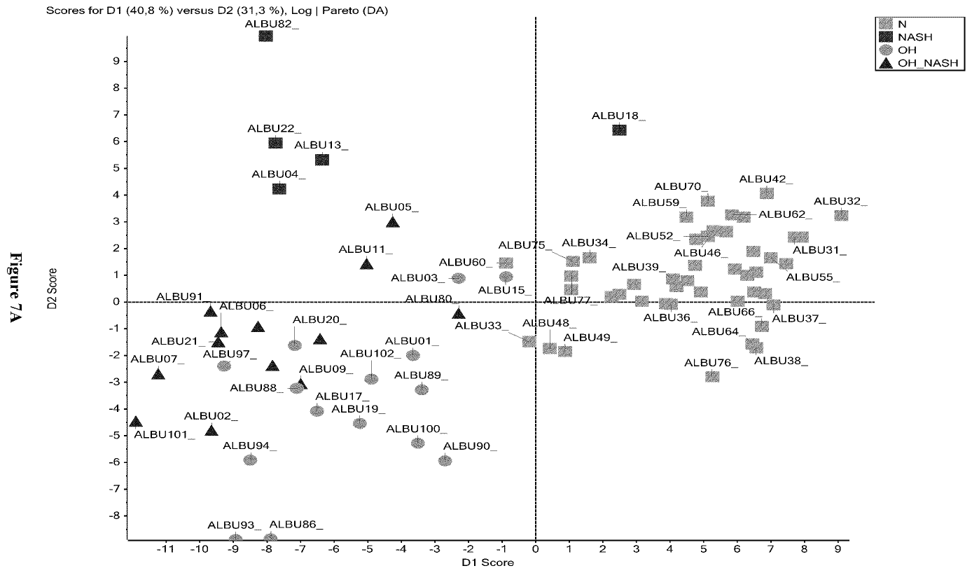

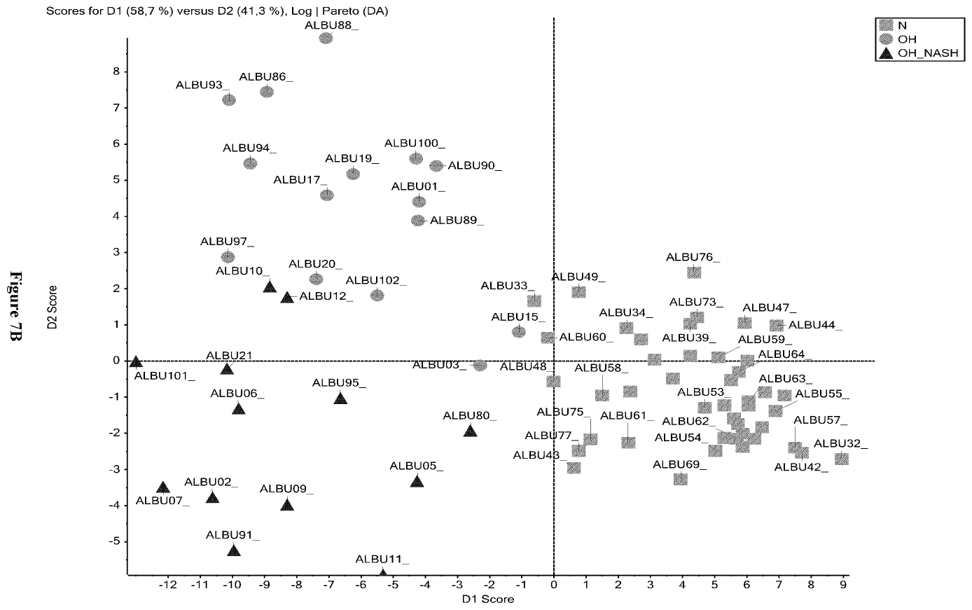

Albumin isoforms were characterized for 38 cirrhotic patients and 52 control patients (with no liver injuries). Among the cirrhotic patients, 18 were diagnosed with alcoholic liver disease (ALD), 5 had a nonalcoholic steatosis (NASH) and 16 were diagnosed with a mixed origin of ALD and NASH. The isoforms detected are depicted in FIG. 6 where we can observe a decrease of native albumin in all cirrhotic patients. This decrease was the most important in NASH patients. Generally, the increase of the different isoforms was not homogenous among the three groups of patients (Table 5). HSA-DA+Cys and HSA+SO₂H were only significantly increased in patients with cirrhosis of mixed origin. HSA+SGGS and HSA+2Glyc were increased only in NASH patients. When considering the whole profile for each patient, PCA-DA showed a clear clustering between the 4 groups, namely, control patients, NASH patients, ALD patients and the mixed-cirrhosis patients (FIG. 7).

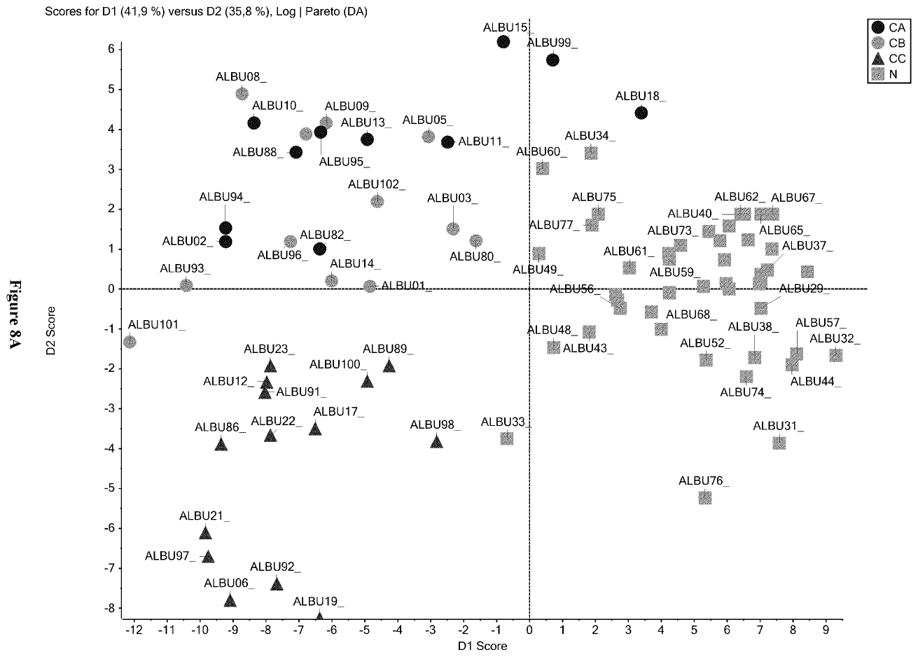

Among the recruited patients, 11 were diagnosed with stage A (calculated on the basis of Child-Pugh score), 12 stage B and 14 stage C cirrhosis. Interestingly, PCA-DA showed a clear clustering of the different stages of cirrhosis and the control patients (FIG. 8).

### Prediction of Early Allograft Dysfunction by the Evolution of the Profiles of Albumin Isoforms:

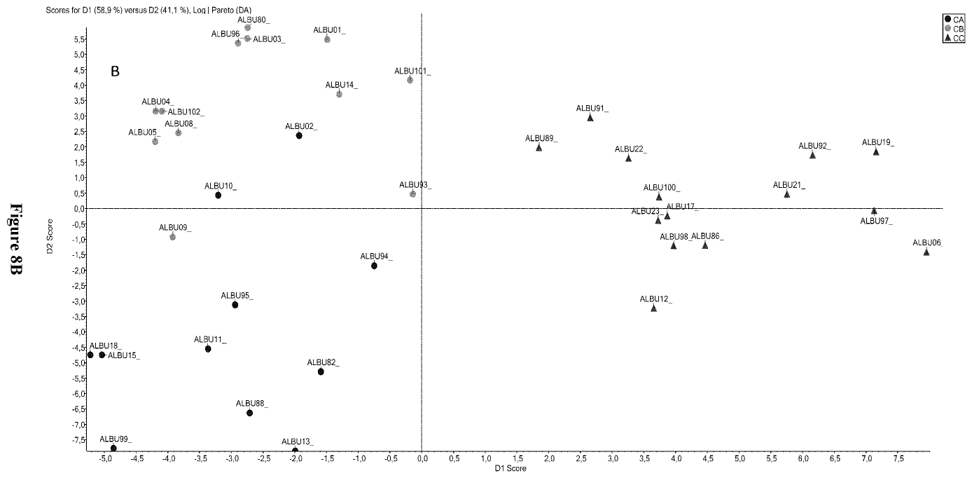

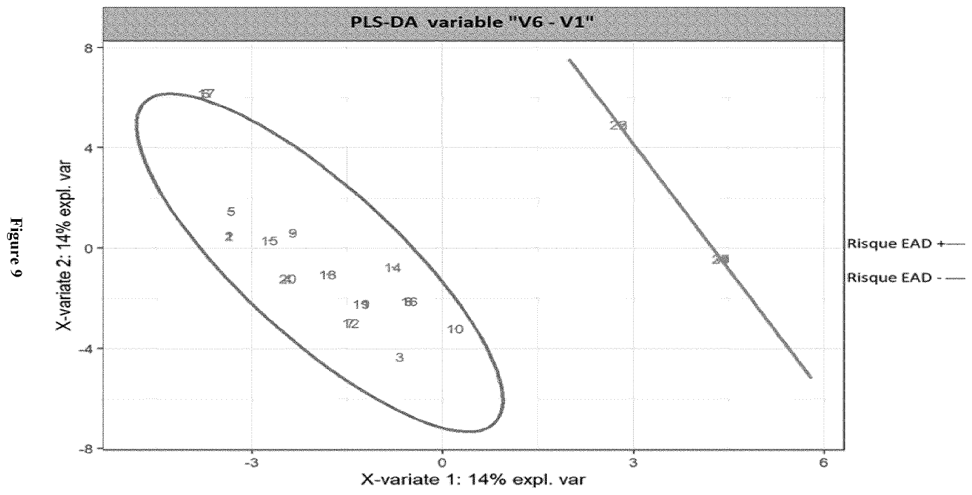

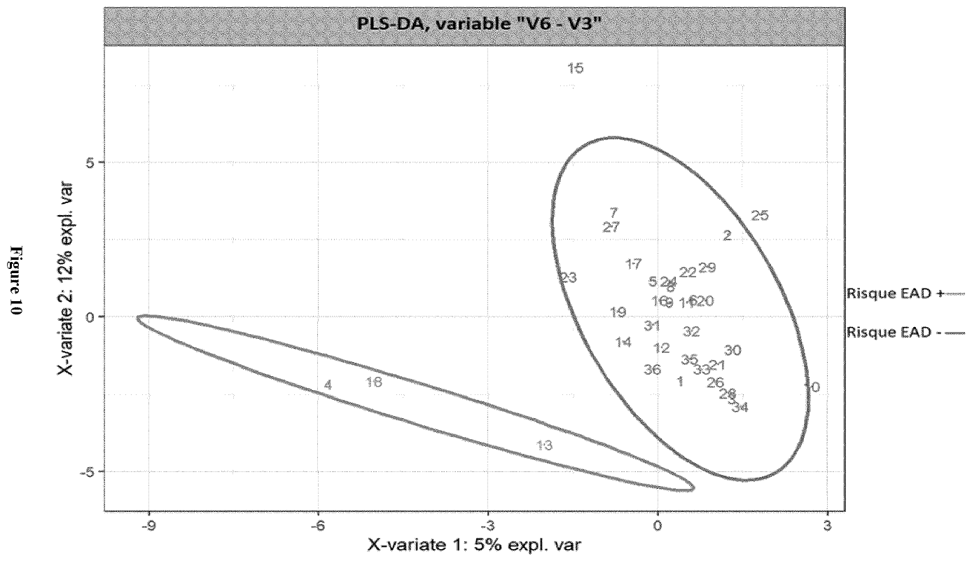

Albumin isoforms were characterized in 38 liver-transplanted patients at different times: V1 (24 h before the transplantation), V2 (during the transplantation), V3 (24 h after the transplantation), V4 (48 h after the transplantation), V5 (72 h after the transplantation), V6 (7 days after the transplantation). Among the 38 patients, 3 experienced an early allograft dysfunction, diagnosed clinically and objectivized by the LGrAFT10 or the EAD scores. Although, albumin isoforms profiles, when taken individually at each time, were not able to discriminate patients with an early allograft dysfunction, the subtraction of the profiles obtained at V6-V1 or V6-V3 allowed a clear clustering of these patients as shown in FIG. 9 and FIG. 10 respectively.

### Discussion

### Precocity of Albumin Modifications:

Our results demonstrate that albumin posttranslational modifications (Alb-PTM) occur very early during the course of liver injuries induced by hepatotoxic substances. In 3 animal models, native albumin started to decrease in favor of other isoforms 24 hours after the administration of APAP, ethanol or CCl₄. Interestingly, the nature and the intensity of isoforms were different depending on the hepatotoxic substance.

Concerning ethanol, it was already known that the daily dose chosen (50% w/v, a very high daily dose) induces inflammation, focal necrosis and finally perivenular fibrosis, within 4 to 29 weeks-depending on the animal model and the experimental protocol (20, 21). In our experimental conditions, inflammation was detected in a few rats (2 out of 7) 24 h after the first administration of ethanol and no more than 33% of rats had inflammation after 2 weeks of daily administration. However, AST and ALT started to increase significantly at D10. The pathogenesis of alcoholic liver disease (ALD) remains poorly understood but is likely a multihit pathophysiological process (22). Ethanol is mostly metabolized into acetaldehyde by ADH, and to a lesser extent, by CYP2E1. A tertiary pathway for the oxidation of ethanol involves catalase, a peroxisomal enzyme that also catalyses the removal of reactive oxygen species (e.g. H₂O₂). Acute exposure to EtOH leads to an adaptive increase in EtOH metabolism within 2 to 3 hours, in both rodent and human livers. This involves the so-called swift increase in alcohol metabolism (SIAM), defined experimentally as a rapid increase in hepatic alcohol metabolism and mitochondrial respiration after a single high dose of alcohol. High demand for 02 during SIAM leads to zones of hypoxia, especially in pericentral (centrilobular) regions of liver lobules, which may contribute to liver injury. Interestingly, the very rapid posttranslational modifications of albumin in our EtOH model are consistent with the rapid onset liver injury described here.

Other mechanisms are at play in longer-term EtOH induced liver injury. EtOH changes the gut microbiome, causing bacterial overgrowth and increasing formation of toxic/proinflammatory products. EtOH consumption also promotes hepatic ROS and RNS formation. EtOH increases CYP2E1, largely by a posttranscriptional mechanism involving stabilization against proteolysis. CYP2E1 generates superoxide (O₂·⁻), which then forms highly reactive peroxynitrite (ONOO⁻) by reaction with NO, and hydroxyl radical (·OH) by the Fenton reaction. In the presence of EtOH, the 1-hydroxyethyl radical is also formed. Therefore, ROS, RNS and other radical species increase after EtOH ingestion. These radicals attack and damage proteins, lipids, and DNA, induce mitochondrial permeability transition (MPT), cause cell death, and trigger inflammatory processes.

Since albumin is the most abundant protein produced in hepatocytes and due to its great reactivity, it is naturally the most prone to posttranslational modifications due to the above-described reactions, which leads to the increase of albumin isoforms as observed in our ethanol model. However, it is still difficult to explain the increase of each isoform individually, since the chemical environment and the reactions at stake are not yet fully understood.

CCl₄ is metabolized in the liver by CYP2E1 and CYP2B1 enzymes and is converted into a highly reactive trichloromethyl (CCl₃·) radical, ultimately leading to hepatotoxic damage, inflammation and fibrosis within 6 to 12 weeks. In our CCl₄ model, steatosis was detected in all rats after 3 days of daily administration. AST and ALT were significantly increased as soon as D1, and BILID after 7 days. The CCl₃· radical, produced after exposure to CCl₄, can bind to cellular molecules (nucleic acid, protein, lipid), impairing crucial cellular processes such as lipid metabolism, with the potential outcome of fatty degeneration leading to steatosis. CCl₃. reacts with oxygen to form the trichloromethylperoxy radical CCl₃OO·, another highly reactive species. CCl₃OO· initiates a chain reaction of lipid peroxidation, which attacks and destroys polyunsaturated fatty acids, in particular those associated with phospholipids. These affect the permeability of mitochondrial, endoplasmic reticulum, and plasma membranes. At the molecular level, CCl₄ activates tumor necrosis factor (TNF)alpha, nitric oxide (NO), and transforming growth factors (TGF)-alpha and -beta in the cell, processes that appear to direct the cell primarily toward (self-)destruction or fibrosis (23).

Despite differences in cellular chemical reactions and environment involved in the mechanism of toxicity of EtOH and CCl₄, the albumin isoforms that we were able to identify in both models were the same with only 3 exceptions: Alb+SO₃H+2Glyc was specific of CCl₄ and Alb+CysO₃ and Alb+Cys+Glyc were specific of EtOH model.

In both models, after a decrease, native albumin increased to reach the level of the control groups at D7, before dropping again. This could be explained by a short regeneration phase, which has been extensively reported for CCl₄ animal models but much less for EtOH (24). Paracetamol is metabolized by CYP2E1 and 1A2 to form -acetyl-p-benzoquinone imine (NAPQI). High concentrations of NAPQI deplete cellular glutathione stores and subsequently form APAP protein adducts, especially with mitochondrial proteins. NAPQI is very reactive and enhances the generation of free radicals such as superoxide. This reacts with nitric oxide (NO) within the mitochondria to produce highly reactive peroxynitrite, which nitrates mitochondrial proteins such as manganese superoxide dismutase (MnSOD). This alters mitochondrial antioxidant defenses, causing mitochondrial oxidant stress and oxidation of proteins such as mitochondrial thioredoxin. In the cytosol, oxidation of thioredoxin results in its detachment from its binding partner apoptosis signal-regulating kinase 1 (ASK1), which is then activated. ASK1, along with activated mixed-lineage kinase 3 (MLK3) then activate c-jun N-terminal kinase (JNK) to its phosphorylated form through MKK4 phosphorylation. Phosphorylated INK translocates to the mitochondria and binds to Sab on the outer mitochondrial membrane, which, through a Src-mediated pathway, further inhibits mitochondrial electron transport. This amplifies mitochondrial oxidant stress, which is further exacerbated by translocation of Bax and glycogen synthase kinase-3β (GSK-3β) from the cytosol to the mitochondria. These events activate the mitochondrial permeability transition, which releases mitochondrial intermembrane proteins such as endonuclease G and apoptosis-inducing factor (AIF), along with cytochrome c and Smac. Translocation of AIF and endonuclease G to the nucleus then induces nuclear DNA fragmentation, which, along with activation of receptor-interacting protein kinases 3/1 (RIP3/RIP1), finally induce programmed necrosis. The extensive cell necrosis after an APAP overdose leads to release of damage-associated molecular patterns (DAMPs) including mitochondrial DNA, nuclear DNA fragments, high-mobility group box 1 (HMGB1) protein, and many others. DAMPs bind to pattern recognition receptors such as toll-like receptors (TLRs) on inflammatory cells and transcriptionally activate cytokine formation in inflammatory cells (25). APAP hepatotoxicity is a time-dependent event involving a number of different phases critical for the injury and recovery process: i) the metabolism phase that includes NAPQI production and glutathione depletion occurs within 0 to 3 hours after APAP administration; ii) the early injury phase that includes JNK activation and mitochondrial translocation, mitochondrial BAX translocation, mitochondrial superoxide formation, MPT, glutathione recovery and ALT/AST increase in plasma occurs within 2 to 6 h; iii) the late injury/early recovery phase that includes ALT/AST increase in plasma, necrosis, innate immune response occurs within 12 to 24h; and finally iv) the regeneration phase where resolution of necrosis could be encountered occurs within 24 to 96 h (26). In our APAP model, necrosis was mainly observed in rats given 4 g/kg APAP, and inflammation was noted for all doses. Native albumin in this model did not decrease 24 hours post APAP and was even barely detectable at 72h, which is consistent with the mechanism of APAP toxicity described here. The sharp decrease of native albumin was associated with an increase in other isoforms. Among them, Alb+SGGS was specific to this APAP model. As we did not observe any normalization of native albumin, we can assume that the regeneration phase was not reached in our experimental conditions.

Overall, these preliminary results support the hypothesis that albumin posttranslational modifications (Alb-PTM) occur early in the course of induced liver injury, suggesting that the use of Alb-PTM as an early biomarker to characterize drug-induced injury would be relevant.

### Albumin Profile as a Signature

Although most of the isoforms characterized in this research are the result of oxidative reactions, albumin can undergo several chemical modifications on several sites of its amino acids chain, including: cysteinylation, homocysteinylation, glutathionylation, glycation, nitrosylation, nitration, oxidation and carbonylation. With our analytical method, we were able to characterize, after the deconvolution of the MS spectra, up to 17 isoforms (Table 4). Our analytical strategy, which could be seen as a simplified top-down analysis, is based on the simple injection of diluted serum into the LC-MS instrument. It has the advantage of preserving the structural and chemical modifications of interest in the native form of the protein. Theoretically, the deconvoluted MS spectrum is capable of identifying unknown modifications because the addition or subtraction of a structural modification result in a characteristic mass shift, equal to the mass of the specific modification (for example, a mass difference of 163 Da for a glycation). However, the 17 isoforms identified had to be “visually detectable” to be included, increasing the risk of leaving behind minor isoforms with low signals. Therefore, to investigate the differences in isoform distribution among the 3 animal models, we decided to integrate the whole information from each spectrum in PCA-DA. The profiles obtained with the 3 models were nicely separated (FIG. 5), suggesting that the Alb profile of each model depends on the different chemical environment and mechanisms of intracellular reactions generated by each substance, as previously explained.

Interestingly, the same approach allowed us to demonstrate that the albumin profiles observed in cirrhotic patients were able to discriminate them from control patients and to discriminate the origins and stages of liver injury among them (FIGS. 7 and 8). This suggests that these isoform profiles may be used as a very valuable tool in the diagnosis and monitoring of liver diseases. In the clinical arena, it is frequently of paramount importance to know whether steatohepatitic liver injury is related to alcohol or NAFLD because this distinction may influence patient management and candidacy for liver transplantation (27).

In our cohort of cirrhotic patients, we could identify up to 14 albumin isoforms, all of which were also present in control patients. However, we observed that the increase in the HSA-DA isoform was specific to patients with cirrhosis due to alcohol abuse, HSA+SGGS and HSA+2Glyc were increased specifically in NASH patients, and HSA-DA+Cys with HSA-SO₂H were increased only in patients with the mixed form. Despite these specificities, the use of the whole information from the MS spectra should increase the discrimination performance of our tool as described above. In addition, we did not observe a specific isoform able to clearly discriminate the different stages of liver disease (data not shown), but principal component analysis of the MS dataset perfectly separated cirrhosis patients with different Child-Pugh scores and control patients.

Overall, albumin isoform profiles represent rich data that could help diagnosing liver diseases, staging them and sorting out their origin.

### Prediction of Early Allograft Dysfunction by the Evolution of the Profiles of Albumin Isoforms:

The rationale for using the albumin isoforms profiles at 2 different times was to compare the evolution and distribution of isoforms between V6 and V3 or V1. The evolutionary nature of this criterion in relation to a baseline situation was intended to use albumin isoform analysis as a functional test, with changes in isoform proportions increasing as the risk of allograft dysfunction increased and graft function was compromised. This is consistent with the fact that the production of less modified albumin after transplantation reflects good graft function. However, because of the low number of patients experiencing an early allograft dysfunction, this observation has to be confirmed in a larger cohort of liver transplanted patients.

### Tables

TABLE 1
Effect of EtOH on biochemical markers expressed as the median [min-max] (* p < 0.05).

| Biomarkers | Controls | D1 | D3 | D7 | D10 | D14 |
|---|---|---|---|---|---|---|
| ALB (g/L) | 14.4 | 13.8 | 12.4* | 12.2* | 16.35 | 12.9* |
| | [12.7-17.6] | [13.2-14.1] | [11.8-14.2] | [11.0-13.8] | [12.4-17.7] | [10.2-13.5] |
| AST (UI/L) | 70.8 | 78 | 80 | 96* | 92* | 90* |
| | [61-75] | [70-85] | [52-144] | [75-157] | [81-157] | [85-294] |
| ALT (UI/L) | 57 | 63 | 62 | 69 | 78* | 82* |
| | [46-61] | [52-71] | [46-123] | [52-97] | [68-98] | [65-104] |
| PAL (UI/L) | 196 | 232 | 180 | 159 | 172 | 230 |
| | [101-325] | [153-304] | [121-329] | [84-263] | [100-222] | [110-327] |
| BILID (μM) | 0.75 | 0.7 | 0.7 | 0.7 | 0.8 | 0.6 |
| | [0.6-1] | [0.3-1] | [0.5-1.3] | [0.4-4.4] | [0.5-1.1] | [0.4-0.7] |
| BILIT (μM) | 0.75 | 0.8 | 0.8 | 1.0 | 1.0 | 0.9 |
| | [0.4-1.3] | [0.4-1.6] | [0.2-1.7] | [0.2-1.9] | [0.8-1.6] | [0.5-1.8] |
| Histology | | 2/7 | 1/6 | 4/7 | 2/8 | 3/9 |
| | | Inflammation | inflammation | inflammation | inflammation | inflammation |

TABLE 3
Effect of CCl₄ on administration on biochemical markers expressed as the median
[min-max] (*p < 0.05; **p < 0.01).

| Biomarkers | Controls | D1 | D3 | D7 | D10 |
|---|---|---|---|---|---|
| ALB (g/L) | 14.4 | 14.2 | 13.0* | 12.9* | 16.0 |
| | [12.7-17.6] | [12.9-14.5] | [11.8-14.2] | [10.4-14.5] | [15.9-16.7] |
| AST (UI/L) | 70.8 | 123* | 151* | 232** | 337** |
| | [61-75] | [90-251] | [82-214] | [168-305] | [192-473] |
| ALT (UI/L) | 57 | 77* | 90* | 200** | 355** |
| | [46-61] | [67-181] | [78-208] | [107-638] | [166-538] |
| PAL (UI/L) | 196 | 232 | 237 | 248 | 205 |
| | [101-325] | [192-270] | [174-329] | [117-341] | [149-250] |
| BILID (μM) | 0.75 | 1.1 | 1.2 | 1.6** | 2.5** |
| | [0.6-1.0] | [0.7-1.5] | [0.3-1.3] | [1.2-2.0] | [1.8-2.9] |
| BILIT (μM) | 0.75 | 1.4 | 1.3 | 2.3** | 3.5** |
| | [0.4-1.3] | [0.4-2.0] | [0.6-3.5] | [1.8-4.5] | [2.4-5.3] |
| Histology | | | Steatosis 5/5 | Steatosis 6/6 | 6/6 steatosis |
| | | | 1/5 fibrosis | | 1/6 fibrosis |

TABLE 3
Effect of APAP administration on biochemical markers expressed as the median [min-max]
(* p < 0.05; ** p < 0.01) and histology examination. D2g-D1 is the group of
rats that were sacrificed 24 h after receiving a dose of 2 g/kg of APAP. D2g-D3 is the
group of rats that were sacrificed 72 h after receiving a dose of a dose of 2 g/kg. D3g-D1
is the group of rats that were sacrificed 24 h after receiving 3 g/kg of APAP.

| Biomarkers | Controls | D2g-D1 | D2g-D3 | D3g-D1 | D3g-D3 | D4g-D1 | D4g-D3 |
|---|---|---|---|---|---|---|---|
| ALB (g/L) | 13.3 | 12.65 | 11.2* | 10.4** | 11.1* | 10.9* | 10.95* |
| | [12.6-14] | [10.6-14.3] | [8.3-12.3] | [9.6-11.8] | [9.9-11.4] | [10.4-12] | [10.2-12.0] |
| AST (UI/L) | 54.35 | 205.2* | 75.95 | 87.1 | 44.3 | 198.8 | 62.9 |
| | [49.5-58] | [113-803] | [31.8-259] | [67-100.2] | [38-95] | [49.1-600] | [56-103] |
| ALT (UI/L) | 34 | 53 | 24 | 31 | 32.5 | 44.5 | 25 |
| | [30-36] | [41-376] | [6-89] | [28-51] | [18-44] | [26-134] | [17-28] |
| PAL (UI/L) | 101 | 134 | 122.5 | 106 | 103.5 | 149 | 96 |
| | [82-113] | [89-243] | [90-157] | [72-167] | [73-142] | [109-241] | [88-111] |
| BILID (μM) | 0.6 | 0.5 | 0.34 | 0.1 | 0.2 | 0.18 | 0.3 |
| | [0.04-1.2] | [0.2-1.1] | [0.1-0.99] | [0.1-1.5] | [0.15-0.6] | [0.08-0.5] | [0.13-0.35] |
| BILIT (μM) | 0.7 | 0.75 | 0.4 | 0.3 | 0.5 | 0.3 | 0.5 |
| | [0.1-1.8] | [0.3-1.4] | [0.1-1.8] | [0.2-2.5] | [0.3-0.9] | [0.2-0.9] | [0.4-3] |
| Histology | | 1/necrosis | 1/6 necrosis | 3/6 portal | 1/6 portal | 5/6 necrosis | 4/6 portal |
| | | | 1/6 | dilatation | dilatation | | inflammation |
| | | | inflammation | 2/6 | 1/6 F1 | | 3/6 F1 |
| | | | 2/6 F1 | inflammation | | | |

TABLE 4
Isoforms of albumin and mass shifts of native albumin in rats and humans.

| Isoformes ID | Rats | Humans | Mass shift | ISOFORMES | APAP | EtOH | CCl4 |
|---|---|---|---|---|---|---|---|
| A | 65685 | 66253 | −185 | HSA-DA | 0 | 0 | 0 |
| B | 65803 | 66371 | −67 | HSA-DA + Cys | 0 | 0 | 0 |
| C | 65870 | 66438 | 0 | HSA Native | 1 | 1 | 1 |
| D | 65901 | 66469 | 31 | HSA + SO2H | 1 | 1 | 1 |
| E | 65918 | 66486 | 48 | HSA + CysO3 | 1 | 1 | 0 |
| F | 65966 | 66534 | 96 | HSA + Cys-DHA | 1 | 1 | 1 |
| G | 65989 | 66557 | 119 | HSA + Cys | 1 | 1 | 1 |
| H | 66018 | 66586 | 148 | HSA + Cys + SNO | 1 | 1 | 1 |
| I | 66033 | 66601 | 163 | HSA + Glyc | 0 | 1 | 1 |
| J | 66065 | 66633 | 195 | HSA + SO2H + Glyc | 1 | 1 | 1 |
| K | 66081 | 66649 | 211 | HSA + SO3H + Glyc | 1 | 1 | 1 |
| L | 66118 | 66686 | 248 | HSA + Cys + Glyc-DHA | 0 | 1 | 1 |
| M | 66152 | 66720 | 282 | HSA + Cys + Glyc | 0 | 1 | 0 |
| N | 66175 | 66743 | 305 | HSA + SGGS | 1 | 0 | 0 |
| O | 66196 | 66764 | 326 | HSA + 2Glyc | 1 | 1 | 1 |
| P | 66242 | 66810 | 372 | HSA + SO3H + 2Glyc | 1 | 0 | 1 |
| Q | 66310 | 66878 | 440 | HSA + Cys + 2Glyc | 1 | 1 | 0 |

TABLE 5
Variations of the different albumin isoforms in the different groups
of patients as compared to the control group.

| | Controls | EtOH | NASH | EtOH-NASH |
|---|---|---|---|---|
| HSA-DA | 0.1% | [symbol] | −0.07% | [symbol] |
| | | 0.4% | | 0.03% |
| HSA-DA + Cys | 0.14% | −0.14% | −0.08% | [symbol] [symbol] [symbol] |
| | | | | 0.5% |
| HSA Native | 37% | [symbol] | [symbol] [symbol] | [symbol] |
| | | 26% | 14% | 29% |
| HSA SO2H | 0.3% | x | x | [symbol] [symbol] [symbol] |
| | | | | 2% |
| HSA SO3H | 3% | −1.6% | x | −3.5% |
| | | | 0.2% | |
| HSA + Cys-DHA | 1% | −1% | [symbol] * | −0.8% |
| | | | 0.5% | |
| HSA + Cys | 27% | [symbol] [symbol] | [symbol] [symbol] | [symbol] [symbol] |
| | | 42% | 47% | 47% |
| HSA + Cys + SNO | 4.7% | [symbol] | [symbol] | [symbol] |
| | | 6.8% | 6.7% | 8.4% |
| HSA + Glyc | 10% | −10% | −10% | −10% |
| HSA + SO2H + Glyc | 1% | [symbol] * | [symbol] * | −1% |
| | | 0.7% | 0.6% | |
| HSA + Cys +Glyc | 8% | −7% | [symbol] [symbol] | −8% |
| | | | 18% | |
| HSA + SGGS | 5% | 5% | [symbol] * | 5% |
| | | | 8.5% | |
| HSA + 2Glyc | 0.4% | [symbol] | [symbol] | [symbol] |
| | | 0.15% | 0.7% | 0.14 |
| HSA + Cys + 2Glyc | 0.13% | [symbol] [symbol] | [symbol] [symbol] [symbol] | [symbol] [symbol] |
| | | 0.4% | 2% | 0.3 |

### REFERENCES

Throughout this application, various references describe the state of the art to which this invention pertains. The disclosures of these references are hereby incorporated by reference into the present disclosure.
1. Elpek G O. Cellular and molecular mechanisms in the pathogenesis of liver fibrosis: An update. World J Gastroenterol 2014; 20:7260-7276.
2. Veyel D, Wenger K, Broermann A, Bretschneider T, Luippold A H, Krawczyk B, Rist W, et al. Biomarker discovery for chronic liver diseases by multi-omics—a preclinical case study. Sci Rep 2020; 10:1314.
3. Wong V W, Adams L A, de Ledinghen V, Wong G L, Sookoian S. Noninvasive biomarkers in NAFLD and NASH—current progress and future promise. Nat Rev Gastroenterol Hepatol 2018; 15:461-478.
4. Zhou J H, Cai J J, She Z G, Li H L. Noninvasive evaluation of nonalcoholic fatty liver disease: Current evidence and practice. World J Gastroenterol 2019; 25:1307-1326.
5. Lin B, Ma Y, Wu S, Liu Y, Liu L, Wu L. Novel Serum Biomarkers for Noninvasive Diagnosis and Screening of Nonalcoholic Fatty Liver Disease-Related Hepatic Fibrosis. OMICS: A Journal of Integrative Biology 2019; 23:181-189.
6. Baldassarre M, Domenicali M, Naldi M, Laggetta M, Giannone F A, Biselli M, Patrono D, et al. Albumin Homodimers in Patients with Cirrhosis: Clinical and Prognostic Relevance of a Novel Identified Structural Alteration of the Molecule. Sci Rep 2016; 6:35987.
7. Baldassarre M, Naldi M, Zaccherini G, Bartoletti M, Antognoli A, Laggetta M, Gagliardi M, et al. Determination of effective albumin in patients with decompensated cirrhosis: clinical and prognostic implications. Hepatology 2021.
8. Das S, Maras J S, Hussain M S, Sharma S, David P, Sukriti S, Shasthry S M, et al.
Hyperoxidized albumin modulates neutrophils to induce oxidative stress and inflammation in severe alcoholic hepatitis. Hepatology 2017; 65:631-646.
9. Domenicali M, Baldassarre M, Giannone F A, Naldi M, Mastroroberto M, Biselli M,
Laggetta M, et al. Posttranscriptional changes of serum albumin: clinical and prognostic significance in hospitalized patients with cirrhosis. Hepatology 2014; 60:1851-1860.
10. Jalan R, Schnurr K, Mookerjee R P, Sen S, Cheshire L, Hodges S, Muravsky V, et al. Alterations in the functional capacity of albumin in patients with decompensated cirrhosis is associated with increased mortality. Hepatology 2009; 50:555-564.
11. Kawakami A, Kubota K, Yamada N, Tagami U, Takehana K, Sonaka I, Suzuki E, et al. Identification and characterization of oxidized human serum albumin. A slight structural change impairs its ligand-binding and antioxidant functions. FEBS J 2006; 273:3346-3357.
12. Klammt S, Mitzner S, Stange J, Brinkmann B, Drewelow B, Emmrich J, Liebe S, et al. Albumin-binding function is reduced in patients with decompensated cirrhosis and correlates inversely with severity of liver disease assessed by model for end-stage liver disease. Eur J Gastroenterol Hepatol 2007; 19:257-263.
13. Naldi M, Baldassarre M, Domenicali M, Bartolini M, Caraceni P. Structural and functional integrity of human serum albumin: Analytical approaches and clinical relevance in patients with liver cirrhosis. J Pharm Biomed Anal 2017; 144:138-153.
14. Oettl K, Birner-Gruenberger R, Spindelboeck W, Stueger H P, Dorn L, Stadlbauer V, Putz-Bankuti C, et al. Oxidative albumin damage in chronic liver failure: relation to albumin binding capacity, liver dysfunction and survival. J Hepatol 2013; 59:978-983.
15. Spinella R, Sawhney R, Jalan R. Albumin in chronic liver disease: structure, functions and therapeutic implications. Hepatol Int 2016; 10:124-132.
16. Stauber R E, Spindelboeck W, Haas J, Putz-Bankuti C, Stadlbauer V, Lackner C, Oettl K. Human nonmercaptalbumin-2: a novel prognostic marker in chronic liver failure. Ther Apher Dial 2014; 18:74-78.
17. Fanali G, di Masi A, Trezza V, Marino M, Fasano M, Ascenzi P. Human serum albumin: from bench to bedside. Mol Aspects Med 2012; 33:209-290.
18. Alcaraz-Quiles J, Casulleras M, Oettl K, Titos E, Flores-Costa R, Duran-Guell M, Lopez-Vicario C, et al. Oxidized Albumin Triggers a Cytokine Storm in Leukocytes Through P38 Mitogen-Activated Protein Kinase: Role in Systemic Inflammation in Decompensated Cirrhosis. Hepatology 2018; 68:1937-1952.
19. Casulleras M, Flores-Costa R, Duran-Güell M, Alcaraz-Quiles J, Sanz S, Titos E, López-Vicario C, et al. Albumin internalizes and inhibits endosomal TLR signaling in leukocytes from patients with decompensated cirrhosis. Sci Transl Med 2020; 12.
20. D'Souza El-Guindy N B, Kovacs E J, De Witte P, Spies C, Littleton J M, de Villiers W J, Lott A J, et al. Laboratory models available to study alcohol-induced organ damage and immune variations: choosing the appropriate model. Alcohol Clin Exp Res 2010; 34:1489-1511.
21. Nevzorova Y A, Boyer-Diaz Z, Cubero F J, Gracia-Sancho J. Animal models for liver disease—A practical approach for translational research. J Hepatol 2020; 73:423-440.
22. Zhong Z, Lemasters J J. A Unifying Hypothesis Linking Hepatic Adaptations for Ethanol Metabolism to the Proinflammatory and Profibrotic Events of Alcoholic Liver Disease. Alcohol Clin Exp Res 2018; 42:2072-2089.
23. Weber L W, Boll M, Stampfl A. Hepatotoxicity and mechanism of action of haloalkanes: carbon tetrachloride as a toxicological model. Crit Rev Toxicol 2003; 33:105-136.
24. Moghaddam A P, Eggers J S, Calabrese E J. Evaluation of sex difference in tissue repair following acute carbon tetrachloride toxicity in male and female Sprague-Dawley rats. Toxicology 1998; 130:95-105.
25. Ramachandran A, Jaeschke H. Acetaminophen Toxicity: Novel Insights Into Mechanisms and Future Perspectives. Gene Expr 2018; 18:19-30.
26. Jaeschke H, Adelusi O B, Akakpo J Y, Nguyen N T, Sanchez-Guerrero G, Umbaugh D S, Ding W X, et al. Recommendations for the use of the acetaminophen hepatotoxicity model for mechanistic studies and how to avoid common pitfalls. Acta Pharm Sin B 2021; 11:3740-3755.
27. Dunn W, Angulo P, Sanderson S, Jamil L H, Stadheim L, Rosen C, Malinchoc M, et al. Utility of a new model to diagnose an alcohol basis for steatohepatitis. Gastroenterology 2006; 131:1057-1063.

1. A method of determining the etiology and severity of a liver injury in a subject and treating the subject, comprising
measuring a level of each of a plurality of albumin isoforms in a blood sample obtained from the subject to establish a profile of albumin isoforms,
determining that the profile is different than a reference profile from subjects without the liver injury, and
treating a subject having profile that is different from the reference profile with surgery and/or by administration of corticosteroids, pentoxifylline, N-acetylcysteine, an antiapoptotic drug or a vasoactive drug.

2. (canceled)

3. (canceled)

4. The method of claim 1, wherein the plurality of isoforms comprises Alb+SO₂H, HSA-CysO₃, Alb+Cys-DHA, Alb+Cys, Alb+Cys+SNO, Alb+SO₂H+Glyc, Alb+SO₃H+Glyc, HAS-SGGS, Alb+2Glyc, Alb+SO₃H+2Glyc, and Alb+Cys+2Glyc.

5. The method of claim 1 comprising i) determining the profile of albumin isoforms in the blood sample obtained from the patient, and ii) comparing the profile to one or more reference profiles associated with various liver injuries.

6. The method of claim 1 wherein the liver injury is a chemical liver injury, a physical liver injury, an ischemic liver injury, an early liver injury, and/or early graft dysfunction or non-function in liver transplanted patients.

7. The method of claim 6 wherein the liver injury is associated with non-alcoholic fatty liver disease.

8. The method of claim 6 wherein the liver injury is liver fibrosis.

9. The method of claim 1, wherein the method predicts the worsening of a liver injury by determining an evolution of a plurality of profiles of albumin isoforms in a plurality of blood samples obtained from the subject over time.

10. The method of claim 1, wherein the method predicts an early allograft liver dysfunction or liver non-function in a liver-transplanted patient by determining an evolution of a plurality of profiles of albumin isoforms in a plurality of blood samples obtained from the liver-transplanted patient over time.

11. The method according to claim 1, wherein the albumin isoforms are detected by mass spectrometry.

12. The method according to claim 1, wherein the method comprises the use of an algorithm.

13. The method of claim 12, wherein the method is a computer-implemented method comprising applying, on a set of values for each albumin isoforms detected in the subject, a trained model configured to determine the etiology and severity of the liver injury based on the set of values.

14. The method of claim 13, wherein the set of values comprises the relative abundance of each detected albumin isoforms.

15. The method of claim 13, wherein the trained model is preliminarily trained by supervised learning on a training dataset comprising, for a plurality of individuals of a population, etiologies and severities of a liver injury.

16. The method of claim 12 wherein the method comprises the use of a classification algorithm.

17. The method according to claim 1, wherein the method determines whether a subject suffering from the liver injury achieves a response to a therapy.

18. The method of claim 7, wherein the non-alcoholic fatty liver disease is NASH.

19. The method of claim 10, wherein the liver fibrosis caused by cirrhosis.

20. The method of claim 1, wherein the surgery is liver transplantation, gall bladder removal or an operation to treat portal hypertension.
